(19) United States
(12) Patent Application Publication
(10) Pub. No.: US 2001/0043931 A1
(43) Pub. Date: Nov. 22, 2001

(54) Human respiratory syncytial virus

(75) Inventors: Xun Zhao, Somerville, MA (US); Mona Singh, Princeton, NJ (US); Vladimir Malashkevich, Belmont, MA (US); Peter S. Kim, Lexington, MA (US)

Correspondence Address: HAMILTON BROOK SMITH AND REYNOLDS, P.C., TWO MILITIA DR, LEXINGTON, MA, 02421-4799, US

(73) Assignee: Whitehead Institute for Biomedical Research, Cambridge, MA (US)

(21) Appl. No.: 09/730,504

(22) Filed: Dec. 5, 2000

Related U.S. Application Data
(60) Provisional application No. 60/169,327, filed on Dec. 6, 1999.

(51) Int. Cl. C12Q 1/70; A61K 39/12

(52) U.S. Cl. 424/204.1; 435/5

(57) ABSTRACT

A method of identifying a drug that inhibits the RSV membrane fusion machinery, such as the HRSV membrane fusion machinery; drugs that bind the N-helix coiled-coil of HRSV, particularly the N-helix coiled-coil pocket, and, thus, inhibit HRSV infection of cells, such as human cells; methods of inhibiting (partially or totally) HRSV infection of an individual; anitbodies that bind to the N-helix coiled-coil region of HRSV F1; and pharmaceutical compositions comprising HRSV infection neutralizing antibodies.

### RELATED APPLICATION(S)

This application claims the benefit of the filing date of U.S. Provisional Application No. 60/169,327, entitled “Human Respiratory Syncytial Virus”, by Xun Zhao, Peter S. Kim and Vladimir Malashkevich, filed on Dec. 6, 1999. The entire teachings of the above application is incorporated herein by reference.

### GOVERNMENT SUPPORT

The invention was supported, in whole or in part, by grants GM44162 and GM56552. from the National Institutes of Health. The Government has certain rights in the invention.

### BACKGROUND OF THE INVENTION

Human respiratory syncytial viruses (HRSV) are enveloped negative-stranded RNA viruses that belong to the pneumovirus subfamily of the family Paramyxoviridae (Collins et al., Respiratory Syncytial Virus. In Fields Virology Third edit. (Fields, B. N., Knipe, D. M. & Howley, P. M., eds.), pp. 1313-1351. Lippincott—Raven Publishers, Philadelphia (1996)). It is a major cause of severe respiratory infections including bronchiolitis and pneumonia in infants and young children worldwide (Heilman, J Infect Dis. 161(3): 402-6 (1990)). Vaccine development for HRSV has been challenging and problematic (Murphy et al., Virus Res 32(1): 13-36 (1994)). To date, no vaccines or drugs have been demonstrated safe and efficacious, and it is very important to develop new therapeutic measures against HRSV infection.

### SUMMARY OF THE INVENTION

Described herein is the X-ray crystal structure of the trimeric α-helical domain of the F1 component of human respiratory syncytial virus (HRSV) envelope glycoprotein which represents the core of fusion-active F1 of HRSV. Also described herein is Applicants' determination, with reference to the X-ray crystal structure, that certain amino acid residues within the core are essential for interaction of the component peptides and, thus, for F1 activity. The core of fusion-active F1 is composed of a trimer of two interacting peptides. The trimer, thus, comprises six peptides (three of each of the two interacting peptides). The interacting peptides are referred to as a peptide derived from the N—terminal region of the ectodomain of HRSV F1 and a peptide derived from the C—terminal region of the ectodomain of HRSV F1. For convenience, these two types of peptides are also referred to as HRSV N—peptide and HRSV C—peptide, respectively. In one embodiment, the two interacting peptides are referred to as N57 and C45. In another embodiment, the two interacting peptides are referred to as N51 and C39. A stable fusion-active core of the HSRV F1 can comprise a 57-residue peptide (N-57) and a 45-residue peptide (C-45) whose amino acid sequences are presented below. Alternatively, a stable fusion-active core of the HSRV F1 can comprise a 51-residue peptide (N-51) and a 39-residue peptide (C-39) whose amino acid sequences are also presented below. The X-ray crystal structure of the N57/C45 complex is a six-helix bundle in which three N57 helices form an interior, parallel coiled coil and three C45 helices pack in an oblique, anti-parallel manner into a highly conserved, concave, hydrophobic surface at one end of a hydrophobic groove on the N57 trimer surface, referred to herein as a semi-pocket or cavity. It shows striking similarity to the low-pH induced conformation of influenza hemagglutinin (HA) and the receptor-binding induced conformation of HIV gp120/gp41.

Applicants have determined the structural basis for interaction between two peptide fragments of HRSV F1: one peptide fragment derived from the N-terminal region of the ectodomain of F1 and one peptide fragment derived from the C-terminal region of the F1 ectodomain. In one embodiment, the N-terminal peptide fragment, N57, includes amino acid residue 153 through and including amino acid residue 209, numbered according to their position in HSRV F0. The amino acid sequence of the N57 peptide is: AVSKVLHLEGEVNKIKSALLSTNKAVVSLSNGVSVLTSKVLDLKNYIDKQLLPIV NK (SEQ ID NO.: 1). The C-terminal region peptide fragment C45 includes amino acid residue 476 through and including amino acid residue 520, numbered according to their position in HRSV F0. The amino acid sequence of the C45 peptide is: NFYDPLVFPSDEFDASISQVNEKINQSLAFIRKSDELLHNVNAGK (SEQ ID NO.: 2).

In another embodiment, the N-terminal peptide fragment, N51, includes amino acid residue 157 through and including amino acid residue 207, numbered according to their position in HSRV F0. The amino acid sequence of the N51 peptide is: VLHLEGEVNKIKSALLSTNKAVVSLSNGVSVLTSKVLDLKNYIDKQLLPIV (SEQ ID NO.: 3). The C-terminal region peptide fragment C39 includes amino acid residue 482 through and including amino acid residue 520, numbered according to their position in HRSV F0. The amino acid sequence of the C39 peptide is: VFPSDEFDASISQVNEKINQSLAFIRKSDELLHNVNAGK (SEQ ID NO.: 4). The compositions and methods of the present invention are generally described in relation to the N57 and C45 peptides and N57/C45 complex, however, it is understood that the compositions and methods of the present invention also relate to the N51 and C39 peptides and N51/C39 complex.

Three-dimension coordinates for the atoms in the N57/C45 F1 complex are presented herein. They can be used to display the structure of the complex, to design a soluble non-aggregating peptide model of the hydrophobic semi-pocket of RSV F1, and to design molecules (drugs) which interact with F1 and inhibit its activity, such as those that prevent interaction of key components (amino acid residues) of the a-helical domain which represents the core of fusion-active F1.

The highly conserved, deep semi-pockets on the N-peptide coiled-coil trimer that accommodate conserved C-peptide residues are useful targets for the development of new peptidomimetic or small-molecule inhibitors of RSV infection. A method of identifying a drug that is an inhibitor of N57/C45 peptide interaction and, thus, is an inhibitor of the RSV membrane fusion machinery and, as a result, reduces or prevents RSV entry into (infection of) cells is the subject of this invention. In the method, N57 and C45 are combined with a drug to be assessed, under conditions suitable for N57 and C45 to interact (suitable for cavities on the N-peptide coiled-coil trimer to accommodate C-peptide amino acid residues). The resulting combination is maintained under these conditions for sufficient time to permit N57 and C45 to interact (e.g., for sufficient time for N57 and C45 to interact in the absence of the drug being assessed). Whether interaction occurs and/or the extent to which N57 and C45 interact is assessed, using known methods. If N57 and C45 do not interact or interact to a lesser extent in the presence of the drug being assessed than in the absence of the drug, the drug to be assessed is an inhibitor of N57/C45 interaction. Such a drug is an inhibitor of the RSV membrane fusion machinery. Such an inhibitor can be further assessed, using in vitro or in vivo methods, for its ability to reduce or prevent RSV entry into cells.

Work described herein provides, for the first time, an understanding of how the N-terminal peptide and the C-terminal peptide interact. The X-ray structure of the crystallized F1 α-helical domain and information regarding the interactions of these two peptides provide the basis for development of drugs which inhibit respiratory syncytial virus (RSV) infection (e.g., HRSV), such as peptidomimetic or small-molecule inhibitors, using such methods as combinatorial chemistry or rational drug design. In addition, a peptide (also referred to as a fusion protein) which includes a soluble, trimeric coiled coil portion and a portion from the N-peptide region of RSV F1 that includes the amino acid residues which form the semi-pocket or cavity of the N-helix coiled-coil of RSV F1 (the pocket-comprising residues of the N-peptide) can be produced and used to identify molecules or compounds which inhibit RSV F1 function and, thus, RSV entry into cells. The trimeric version of the coiled-coil in the peptide (also referred to as a fusion protein) can be the coiled-coil region of a protein which is not a protein of RSV (a non RSV protein, such as GCN4-pIQI as described in U.S. application Ser. No. 09/364,497 and Eckert, D. M. et al. (1999) Cell 99, 103-115) or a protein of RSV origin (a protein derived from RSV or having the same or a similar amino acid sequence as an RSV protein). Alternatively, this component of the fusion protein can be a trimeric version of the coiled-coil region of another protein, such as that from Moloney Murine Leukemia Virus (Fass, D. et al. Nature Struct. Biology, 3:465 (1996)), GCN4-pII (Harbury et al., Nature, 317:80, 1994) or the ABC heterotrimer (Nautiyal and Alber, Protein Science 8:84 (1999)).

The soluble model (in the natural L-handedness or enantiomeric D-handedness) can be used in screens, including high-throughput drug screens, to identify molecules that bind to the coiled-coil semi-pocket. The soluble model, in the D-handedness, can be used as a target in mirror image phage display (Schumacher et al., Science, 271: 1854, 1996) to identify small molecules (D-peptides) which bind to the hydrophobic semi-pocket of F1 (in the natural L-handedness) and inhibit RSV-membrane fusion. The desired target (the N-helix of RSV F1 which includes the hydrophobic semi-pocket) is the exact mirror image of the naturally-occurring target. It is used to screen a library or collection of compounds or molecules which are to be assessed for their ability to bind the mirror image of the naturally-occurring coiled-coil semi-pocket. The mirror image of a compound or molecule found to bind the mirror image of the naturally-occurring F1 semi-pocket, will bind the F1 pocket in the natural handedness. The library or collection screened can be of any type, such as a phage display library, peptide library, DNA library, RNA library, combinatorial library, collection of chemical agents or drugs, cell lysate, cell culture medium or supernatant containing products produced by cells. In the case of a phage display library, the D-target is used to screen phage coat proteins. Specific phage clones that bind to the target are identified and the mirror images of the expressed proteins are chemically synthesized with D-amino acids. By using the soluble model in mirror-image phage display, D-peptides that bind to the F1 hydrophobic semi-pocket can be identified. Further assessment can be carried out to demonstrate the ability of D-peptides to inhibit RSV F1 function. D-peptides which bind the hydrophobic pocket also will serve as lead molecules for drug development and/or reagents for drug discovery (where the drugs bind to the coiled-coil semi-pocket and inhibit RSV infectivity). The soluble model, in the natural L-handedness, can be used in screens, including high-throughput screens, to identify molecules that bind to the coiled-coil semi-pocket. The soluble model can be used to screen a collection or library of compounds or molecules which are to be assessed for their ability to bind the hydrophobic pocket. The library or collection screened can be of any type, such as a phage display library, RNA library, DNA library, peptide library, combinatorial library, collection of chemical agents or drugs, cell lysate, cell culture medium or supernatant containing products produced by cells. Compounds or molecules which bind the hydrophobic semi-pocket also will serve as lead molecules for drug development and/or reagents for drug discovery.

Fusion proteins which are variants of the soluble model can be produced and used to screen for drugs which bind the F1 N-helix coiled-coil pocket. Any of a wide variety of variations can be made in the soluble model and used in the method, provided that these changes do not alter the trimeric state of the coiled-coil. For example, the amino acid composition can be changed by the addition, substitution, modification and/or deletion of one or more amino acid residues, provided that the trimeric state of the coiled-coil is maintained.

Changes can also be made in the amino acid composition of the fusion protein component which is the C-terminal portion of the RSV F1 N peptide to produce variants of the soluble model. The C-terminal portion can be changed by the addition, substitution, modification and/or deletion of one or more amino acid residues. The amino acid composition of either or both components of the fusion protein can be altered, and there is no limit to the number or types of amino acid residue changes possible, provided that the trimeric state of the coiled-coil and the hydrophobic pocket of the N peptide of RSV F1 are maintained. The soluble model of the F1 semi-pocket can be used to screen for drugs which bind the N-helix coiled-coil, especially the pocket, or for lead drug candidates or candidates for use in vaccine preparations, to be further screened using methods known to those of skill in the art, such as in a high throughput format.

Drugs developed or identified with reference to the information provided herein are also the subject of the present invention (e.g., peptides such as L or D peptides). Drugs that fit into or line the N-peptide semi-pocket, prevent the N-peptide semi-pocket from accommodating amino acid residues or peptides from the C-terminal region of F1. As a result, such drugs prevent or inhibit F1 activity and/or RSV membrane fusion machinery are the subject of this invention. Such drugs can be identified with reference to the information about the structure of the complex and the semi-pocket shown to be present in the N57 trimer, provided herein, or with reference to information about the structure of the complex, and known methods. In a particular embodiment of identifying or designing a molecule which inhibits the fusion active form of F1 and, thus, inhibits RSV, in which combinatorial chemistry is used, a library biased to include an increased number of aromatic rings, hydrophobic moieties and/or polar groups is used.

An antibody (polyclonal and/or monoclonal) which binds these key areas of fusion-active F1 is also the subject of the invention. For example, an immunogen which is or includes a molecule with the coordinates described herein, the N-peptide core and/or the soluble model described herein can be used to immunize an individual, resulting in production of antibodies that bind the cavity or semi-pocket on the N-terminal peptide and, thus, render it unavailable for its normal interactions and prevent or inhibit F1 activity. Thus, a further subject of this invention are compositions (e.g., proteins or proteinaceous materials) that can be used to elicit an immune response (e.g., antibody production) that will protect (partially or totally) against HIV infection and/or disease. Such compositions are useful as protective agents (e.g., vaccines) and to obtain antibodies that are useful as research tools, diagnostic tools, drug screening reagents, and to assess viral dynamics (rates of production and clearance of virus) in animal models or infected humans.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 is a schematic diagram of the HRSV F protein sequence. F1 and F2 are formed after proteolytic cleavage of the precursor F0. The signal peptide (SP), the cleavage site (CS), the putative fusion peptide (FP), the N-terminal heptad-repeat (HR-N) region, the C-terminal heptad-repeat (HR-C) region, and the transmembrane segment (TM) are indicated. The amino acid sequence of the recombinant recRSV-1 construct is indicated. The HR-N and HR-C regions predicted by LeamCoil-VMF are indicated by shaded boxes. Protease-resistant fragments resulting from trypsin (HRSV-N57 and HRSV-C45) and proteinase K (HRSV-N51 and HRSV-C39) cleavage are indicated.

FIG. 2 is a schematic representation of a model for virus-cell membrane fusion.

FIG. 3A is the CD spectra of HRSV-N57, HRSV-C45, and HRSV-N57/C45 in PBS at 25° C. The predicted spectrum for noninteracting HRSV-N57 plus HRSV-C45 peptides is shown for comparison.

FIG. 3B shows the equilibrium sedimentation of HRSV-N57/C45 at 20° C. and 15,000 rpm in PBS. The data fit closely to a trimeric model. Lines expected for dimeric and tetrameric models are indicated for comparison. The deviation in the data from the linear fit for a trimeric model is plotted in the upper panel. No systematic deviation in the residuals is observed.

FIGS. 4A-4C show the crystal structure of HRSV-N57/C45 complex. FIG. 4A shows a region of a simulated annealing omit map of HRSV-N57/C45, calculated with 2Fₒ-F_{c} coefficients in the absence of the fragments shown. The map is displayed as a map cover contoured at 1σ. The view is down the three-fold axis of the trimer. FIG. 4A was generated with the program O (Jones, T. A. et al. (1991) Acta. Crystallogr. D47, 110-119). FIG. 4B is a ribbon diagram of the HRSV-N57/C45 trimer; it shows a top view of the HRSV-N57/C45 trimer looking down the three-fold axis of the trimer. FIG. 4C shows a side view with the amino termini of the N-helices pointing toward the top of the page and those of the C-helices pointing toward the bottom. Figure prepared using MOLSCRIPT (Laskowski, R. A. et al. (1993) J. Appl. Crystallogr. 26, 283-291). N-helices are shown in blue and C-helices in yellow.

FIGS. 5A and 5B show the surface properties of the HRSV-N57 trimer with the HRSV-C45 peptides displayed in a stick-style representation. View same as FIG. 4C. FIG. 5A shows the surface variability of the HRSV-N57 trimer (analysis based on RSV sequences available in GenBank and Swiss-Prot). The residues shown in dark red vary among different human virus strains. The residues shown in pink are identical among 20 human strains of HRSV, but are different in bovine respiratory syncytial virus (BRSV). The cavity region is indicated by an arrow. FIG. 5B shows surface mapping of groups with the potential to form electrostatic and polar interactions. Nitrogen and oxygen atoms from charged amino acid side chains are shown in blue and red, respectively. Nitrogen and oxygen atoms from polar amino acid side chains are shown in yellow. Figure drawn with the program GRASP (Nicholls, A. et al. (1991) Proteins 11, 281-296).

FIGS. 6A and 6B show the cavity on the surface of the HRSV-N57 coiled coil. FIG. 6A is a stereo view of interactions in the HRSV-N57 cavity. Two phenylalanine residues of HRSV-C45 (yellow) fit into the cavity between the two neighboring HRSV-N57 peptides (blue). A prime symbol (′) is used to distinguish residues from two different HRSV-N57 chains. Figure prepared using MOLSCRIPT. FIG. 6B is a comparison of cavity interactions of the HRSV and SV5 structures. The HRSV-N57 coiled coils, which superimpose closely with the SV5 N-peptide coiled coils, are represented as a molecular surface (the most convex part shown in green and the most concave in gray). The HRSV-C45 (yellow) and SV5 C-peptide (pink) helices are shown as ribbons with selected side chains that pack into the cavity. The relative shift of the C-peptides is clearly visible. Figure drawn with the program GRASP.

### DETAILED DESCRIPTION OF THE INVENTION

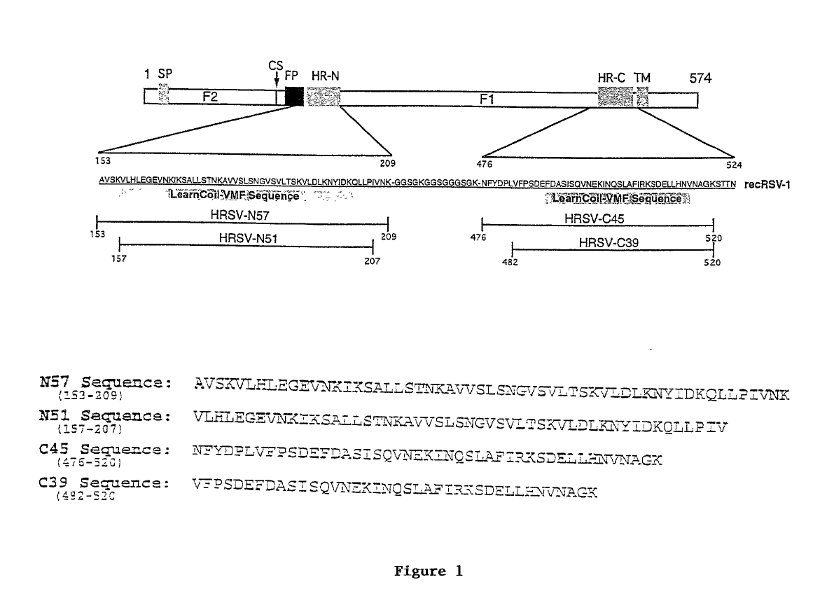

For the first time, a picture of the protein fragment that enables HRSV to invade human cells has been produced. As described, Applicants have crystallized and determined the X-ray structure of a key fragment of F1, an HRSV envelope protein. HSRV carries three surface glycoproteins: F, G and SH (Collins, P. L., et al. (1996) in Fields Virology, Third ed. (Fields, B. N., et al., eds.) pp.1313-1351. Lippincott—Raven Publishers, Philadelphia). The HRSV F protein is an attractive target for drug and vaccine development, as it is essential for viral entry, is highly conserved, and is the major virus neutralization antigen (Collins, P. 1. et al. (1996) in Fields Virology, Third ed. (Fields, B. N. et al. Eds.) pp. 1313-1351. Lippincott—Raven Publishers, Philadelphia). In contrast, the G protein is highly variable among different strains of HRSV (Johnson, P. R. et al. (1987) Proc. Natl. Acad. Sci. USA 84, 5625-5629; Johnson, P. R. and Collins, P. L. (1988) J. Gen. Virol. 69, 2623-2628). The F protein is synthesized as a 67 kDa precursor (denoted F0) that is proteolytically cleaved to two disulphide—linked subunits: F1 (47 kDa) and F2 (21 kDa) (See FIG. 1). The fusion peptide region, a hydrophobic/glycine-rich segment that inserts into the target cellular membrane during the fusion process (Collins, P. L., et al. (1996) in Fields Virology, Third ed. (Fields, B. N., et al., eds.) pp.1313-1351. Lippincott—Raven Publishers, Philadelphia) is located at the N-terminus of the F1 subunit. The transmembrane segment is close to the C-terminus of the F1 subunit. Adjacent to both the fusion peptide and transmembrane segments are two regions containing 4,3 hydrophobic heptad repeats (HR), a sequence motif suggestive of coiled-coil structures (Chambers, P. et al. (1990) J. Gen. Virol. 71, 3075-3080; Buckland, R. & Wild, F. (1989) Nature 338, 547; Singh, M. et al. (1999) J. Mol. Biol. 290, 1031-1041). These regions are denoted HR-N and HR-C, respectively, and are separated by an intervening domain of ˜270 residues. In general, the arrangement of these structural elements in the F1 protein is highly conserved among the Paramyxoviridae family (Lamb, R. A. (1993) Virology 197, 1-11).

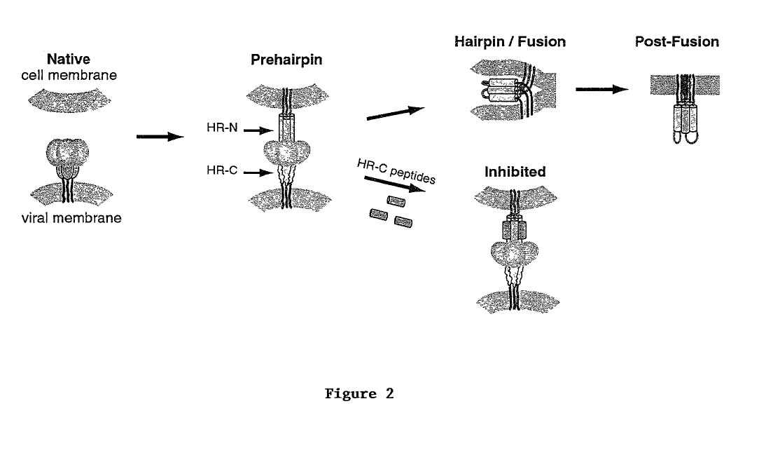

There is extensive biochemical evidence to suggest that viral fusion proteins undergo conformational changes when activated (Hughson, F. M. (1997) Curr. Biol. 7, R565-R569; Hernandez, L. D. et al. (1996) Annu. Rev. Cell Dev. Biol. 12, 627-661). Earlier work on the HIV-1 fusion protein gp41 led to a model for viral membrane fusion (FIG. 2) in which gp41 exists in at least three different conformations: (i) the native (nonfusogenic) form, where the fusion peptide and HR-N region are buried, (ii) the prehairpin intermediate, where the HR-N region is exposed and the fusion peptide is projected into the target cellular membrane, and (iii) the fusogenic hairpin form, where the HR-N and HR-C regions associate and bring the viral and the cellular membranes together to facilitate fusion (Chan, D. C. & Kim, P. S. (1998) Cell 93, 681-684). The model shown is based primarily on studies of the HIV-1 gp41-mediated membrane fusion process. The HRSV F protein likely undergoes similar conformational changes. In the native state, the fusion peptide (not shown) is buried. Upon activation, the fusion protein undergoes a conformational change to the prehairpin intermediate, in which the fusion peptide (red lines) is inserted into the target-cell membrane and the HR-N peptide region (gray) is a trimeric coiled coil. The HR-C peptide region (yellow) has not yet associated with the N-peptide coiled coil. This intermediate is vulnerable to HR-C peptide inhibition (bottom; inhibitory peptides shown in orange). The prehairpin intermediate resolves to the fusion-active hairpin structure when the HR-C peptide region binds to the HR-N peptide coiled coil and adopts a helical conformation. This rearrangement results in membrane apposition. Whether hairpin formation precedes the actual membrane-fusion event or occurs simultaneously with fusion is unknown.

Agents that prevent these conformational changes by stabilizing the native or intermediate states are expected to prevent fusion activation and thus block viral entry. In the case of HIV-1 gp41, peptides corresponding to the HR-C region of gp41, referred to as C-peptides, can effectively inhibit infection (Chan, D. C. & Kim, P. S. (1998) Cell 93, 681-684). One such C-peptide is in clinical trials and shows antiviral activity in humans (Kilby, J. M. et al. (1998) Nat. Med. 4, 1302-1307). C-peptides function in a dominant-negative manner by binding to the transiently exposed coiled coil in the prehairpin intermediate and consequently blocking the formation of the fusion-active hairpin structures, thereby inhibiting viral entry.

A similar approach may be effective in identifying inhibitors of HRSV infection. HR-C regions from HRSV have been shown to inhibit viral infection, and thus provide evidence that the F protein may also display the prehairpin intermediate conformation (Lambert, D. M. et al. (1996) Proc. Natl. Acad. Sci. USA 93, 2186-2191). Analogous to the HIV-1 C-peptides, these HRSV HR-C peptides likely act in a dominant-negative manner to prevent the formation of final fusion-active structures, thereby inhibiting viral entry.

The interaction of the two heptad-repeat regions of HRSV F1 protein were characterized to verify that the HRSV F1 protein core indeed forms a trimer-of-hairpins structure and to provide a structural basis for the development of fusion inhibitors. Two peptides—HRSV-N57 and HRSV-C45, that correspond to the HR-N and HR-C regions of HRSV F1 protein, respectively—were shown to form a stable trimer of heterodimers. This result is consistent with recent reports that indicate the HR-N and HR-C regions from HRSV associate to form an α-helical trimer of heterodimers (Matthews, J. M. et al. (2000) J. Virol. 74, 5911-5920; Lawless-Delmedico, M. K. et al. (2000) Biochemistry 39, 11684-11695). The HRSV-N57/C45 complex characterized here was crystallized and its X-ray structure determined to 2.3 Å resolution. The structure confirms the similarity between the HRSV F1 protein core and several other viral fusion proteins, including HIV-1 gp41, suggesting that methods for discovery of potential therapeutics and prophylactics developed against HIV-1 gp41 might also apply to the HRSV F1 protein.

The HRSV fusion protein has characteristics similar to those of the fusion structures of the influenza virus and the human immunodeficiency virus (HIV). The HRSV fusion protein has a deep groove on the surface of the N57 trimer. In the active structure, the groove is filled by C45. This arrangement provides a target for drug design or discovery. The structure, combined with data from other laboratories, supports the idea that a small molecule constructed specifically to block this interaction will stop fusion and prevent the virus from entering cells.

Such a molecule would be effective in preventing RSV, such as HRSV, from entering cells because peptides corresponding to the C-terminal heptad repeat regions have been shown to inhibit viral infection (Lambert, D. M. et al. (1996) Proc. Natl. Acad. Sci. USA 93, 2186-2191). However, peptides generally make poor drugs because they are poorly absorbed and the body breaks them down almost immediately. A small molecule targeting just the cavity structure could escape this fate.

Applicants have analyzed the X-ray structure of the crystallized α-helical domain of the HRSV transmembrane protein F1 by means of assessment of a complex, referred to herein as the N57/C45 complex, which is composed of two interacting peptides: N57, which is derived from the N-terminal region of the F1 ectodomain and C45, which is derived from the C-terminal region of the F1 ectodomain. As described herein, Applicants have shown that the N57/C45 complex is a six-helix bundle, in which the center consists of a parallel, trimeric coiled-coil of three N57 helices wrapped in a gradual left-handed superhelix. Three C45 helices wrap antiparallel to the N57 helices in a left-handed direction around the outside of the central coiled-coil N57 trimer. The N57/C45 complex forms a rod-shaped trimer-of-hairpins structure. One trimer is approximately 68 Å in length and 27 Å in diameter. The characteristic “knobs-into-holes” packing of coiled coils occurs in the N36 trimer. That is, particular amino acid residues (knobs) of the C45 peptide pack into grooves (holes) formed on the N57 peptide (Crick, F. H. C., Acta. Cryst., 6: 689-697 (1953); O'Shea, E. K., et al., Science, 254:539-544 (1991). As described herein, there are 20 amino acids from the C45 peptide and 26 amino acids from the two N57 peptides that are involved in the interface interaction. These residues are highly conserved among different RSV strains (including twenty HRSV isolates and seven BRSV isolates). In the N-terminal region of C45, residues 483-488 (FPSDEF (SEQ ID NO: 5)) fit into a concave hydrophobic surface at one end of the hydrophobic groove on the N57 trimer surface. This surface is referred to as a semi-pocket since it has well-shaped walls at three sides but flattens into the hydrophobic groove at the fourth side. Both F483 and F488 are almost completely buried by the semi-pocket as judged by a side chain solvent accessibility close to zero. Six N57 residues, K191, L195, and Y198 from one N57 chain and residues K196, D200, and L204 from another N57 chain, line around the semi-pocket. Besides the semi-pocket, there is another patch at the middle of the hydrophobic groove where extensive contacts are observed.

As a result of the work described, a region of the HRSV transmembrane protein F1, which is a target for RSV inhibitors, has been defined and is available for designing and/or developing new drugs and identifying existing drugs which inhibit HRSV. A particularly valuable target for a RSV inhibitor, particularly a HRSV inhibitor, is the highly conserved, semi-pocket on the N-peptide coiled-coil trimer that accommodates C-peptide amino acid residues. The amino acid residues that form the semi-pocket have been defined. Thus, a drug (e.g., a peptide, peptidomimetic, small molecule or other agent) which fits into or lines the N-peptide semi-pocket, prevents the N-peptide cavity from accommodating peptides from the C-terminal region of F1 and, thus, prevents or inhibits F1 activity, can be identified or designed. For example, a drug that fits into or lines the cavity can be identified or designed, using known methods. One such drug is a molecule or compound which fits into a semipocket lined by K191, L195 and Y198 from one (a first) N57 chain and K196, D200 and L204 from another (a second) N57 chain.

As indicated above, the semi-pocket present on the surface of the N57 trimer accommodates amino acid residues abutting from the C45 helix: in the N-terminal region of C45, residues 483-488 fit into a concave hydrophobic surface at one end of the hydrophobic groove on the N57 trimer surface. There is another patch at the middle of the hydrophobic groove where extensive contacts are observed. Two neighboring residues in the helical portions of C45, F505 and I506, contribute 15 and 11 van der Waals contacts, respectively, into the interface. On the preceding helical turn, S502 contributes 14 van der Waals contacts plus one hydrogen bond. On the following helical turn, S509 contributes 11 van der Waals contacts and three hydrogen bonds. In total, the interface between the short peptide segment 502-509 and the N helices involves 52 van der Waals contacts and 4 hydrogen bonds. A drug that mimics the ability of the C45 residues to fit into or line the N57 semipocket can also be developed. Such a drug can be developed, for example, with reference to the three-dimensional structure provided herein.

A structure-based approach can be used, along with available computer-based design programs, to identify or design a drug that will fit into, line or bind a cavity or semi-pocket on N57 (or block C45 from doing so) and inhibit or prevent the activity of F1 and, as a result, reduce (partially or totally) the ability of HRSV to infect cells. In one embodiment of the present invention, the following method is carried out to design or identify a molecule or drug which inhibits F1activity (and reduces HRSV infection of cells) by fitting into or lining the N57 semi-pocket. In a computer processor having a digital processor, a method of designing or identifying a drug or molecule which inhibits (totally or partially) the interaction of N57 and C45 or fits into or lines a semi-pocket on N57, comprises the steps of: (a) providing a library of molecules, compounds or drugs whose crystal structures, coordinates, chemical configurations or structures are known; (b) providing a crystal structure of a target molecule, which is the a-helical domain of the F1 component of HRSV envelope glycoprotein which represents the core of fusion-active F1 (referred to for convenience as the N57/C45 complex or N57/C45); and (c) comparing coordinates, crystal structure components, chemical configurations or structures of members of the library of molecules with those of the target molecule, such as by using a processor routine executed by the digital processor to search the library to find a molecule or a molecule component which fits into or lines the semi-pocket on N57, the processor routine providing design or identification of a member or members of the library which fit into or line the semi-pocket on N57 or a member or members which comprise a component moiety or component moieties which fit into or line the semi-pocket on N57. For example, this method can be carried out by comparing the members of the library with the X-ray crystal structure of F1 N57/C45 presented herein using computer programs known to those of skill in the art (e.g., Dock, Kuntz, I. D. et al. (1992) Science 257, 1078-1082; Kuntz, I. D. et al. (1982) J. Mol. Biol., 161, 269; Meng, E. C., et al. (1992) J. Comp.Chem. 13, 505-524 or CAVEAT).

In the method, the library of molecules to be searched in (a) can be any library, such as a database (i.e., online, offline, internal, external) which comprises crystal structures, coordinates, chemical configurations or structures of molecules, compounds or drugs (referred to collectively as to be assessed or screened for their ability inhibit N57/C45 interaction candidate N45 ligands). For example, databases for drug design, such as the Cambridge Structural Database (CSD), which includes about 100,000 molecules whose crystal structures have been determined or the Fine Chemical Director (FCD) distributed by Molecular Design Limited (San Leandro, Calif.) can be used (CSD: Allen, F. H. et al. (1979) Acta Crystallogr. Section B, 35, 2331). In addition, a library, such as a database, biased to include an increased number of members which comprise aromatic rings, hydrophobic moieties and/or polar groups can be used.

Coordinates of the molecules in the library can be compared in the method to coordinates of the semi-pocket on N57 or to coordinates of C45 and its components that fit into or line an N57 cavity or semi-pocket. The semi-pocket on N57 is described in detail herein, as are key components of C45 that are accommodated by the semi-pocket on the N-peptide. Upon finding a match to coordinates of at least one molecule in the library, at least one member is, thus, determined or identified as an N57 ligand (at least one member is determined to be a member which will inhibit N57/C45 interaction).

Additional steps in the searching process can include combining certain library members or components of library members to form collective coordinates or molecules which combine features or coordinates of two or more library members; comparing the resulting collective coordinates or molecules with the crystal structure of the target molecule and identifying those which will interact with an N57 semi-pocket (or cavitiy).

Upon identification of an existing drug or design of a novel molecule as described herein, its ability to line or fit into a semi-pocket on N57 or block N57/C45 interaction can be assessed using known methods, such as by expressing N57 and C45 in an appropriate host cell (e.g., a bacterial cell containing and expressing DNA encoding N57 and C45), combining the expressed products with the drug to be assessed and determining whether it interferes with the interaction of N57 and C45 and/or lines a semi-pocket formed by two N57 peptides. Drugs which are found to do so can be assessed in additional assays, both in vitro and in vivo (e.g., an appropriate animal model challenged by RSV infection). Once a drug has been identified or designed, it may be desirable to refine or reconfigure it in such a manner that a drug which binds better (e.g., with greater specificity and/or affinity) is produced. In this case, the processor routine further determines the quality of matches and calculates a goodness of fit, making it possible to do so.

In addition, as described herein, hybrids (i.e., fusion proteins) that are trimeric (i.e., not aggregated) and helical (e.g., 100%) can be made between a trimeric version of the coiled-coil region of a protein (such as GCN4) and the N-helix coiled-coil of F1. Therefore, the invention relates to another embodiment of a method of identifying a drug that binds the N-helix coiled-coil cavity of HIV gp41. Here, too, the assay is based on assessing loss or decrease in binding, but unlike the C57/N45 complex assay described above, which is a more general assay in that it covers or detects interaction with any portion of the groove formed by the N-helical region of HRSV F1, this embodiment focuses on the HRSV F1 hydrophobic semi-pocket (the N-helix coiled-coil semi-pocket). In this embodiment, the method comprises combining a candidate drug to be assessed for its ability to bind the N-helix coiled-coil cavity of HRSV F1 with a fusion protein that comprises a trimeric version of the coiled-coil region of a protein and a sufficient portion of the N-peptide of HRSV F1 to include the HRSV F1 semi-pocket, under conditions appropriate for presentation of the HRSV F1 semi-pocket for binding by a peptide or other molecule and determining (e.g., in a high throughput screen) whether the candidate drug binds the fusion protein. If binding occurs, the candidate drug is a “hit” that may be a drug that binds the N-helix coiled-coil semi-pocket of HRSV F1. If binding occurs, the candidate drug has bound the N-helix coiled coil and it can be determined if it binds to the coiled-coil cavity. Such “hits” can then be screened in secondary assays, such as the cell/cell fusion assay and HRSV infectivity assay to determine if the candidate drug is a drug. Alternatively, or in addition, such “hits” can be assessed further by use of a counterscreen with other fusion proteins (or peptides), to which pocket-binding molecules will not bind.

In a further embodiment, a competitive assay is carried out. In this embodiment, a peptide or protein that binds the N-helix coiled-coil cavity of HRSV F1 is combined with the candidate drug and the fusion protein and whether the candidate drug binds the HRSV F1 semi-pocket is determined in the presence of the peptide that binds the N-helix coiled-coil semi-pocket of HRSV F1. If the candidate drug binds the fusion protein, it is a drug that binds the HRSV F1 semi-pocket. For example, a fusion protein which comprises a trimeric version of the coiled-coil region of GCN4 and the C-terminus of the N peptide of HRSV F1 that includes the N-helix coiled-coil cavity is combined with a “reference” D-peptide (e.g., any of the D-peptides described herein or variants thereof) that binds the N-helix coiled-coil cavity and a candidate drug to be assessed for its ability to bind the N-helix coiled-coil semi-pocket of HRSV F1, thus producing a test sample, which is maintained under conditions appropriate for binding of the D-peptide to bind to the cavity. A control sample, which includes the same components as the test sample, except for the candidate drug, and is handled in the same manner as the test sample, is also assessed. In both samples, binding of the reference D-peptide is assessed. If binding of the reference D-peptide occurs to a lesser extent in the presence of the candidate drug (in the test sample) than in its absence (in the control sample), the candidate drug is a drug that binds the N-helix coiled-coil semi-pocket of HRSV F1. Detection of binding is assessed, for example, in a similar manner as described above for the N57/C45 embodiment of the invention. For example, the D-peptide is labeled with a detectable label, such as a radiolabel or a first member of a binding pair (e.g., biotin), and the extent to which the N-helix coiled-coil cavity bears the label (after the samples have been maintained under conditions appropriate for binding of the reference D-peptide to the semi-pocket) is determined. In the case in which radiolabeling is used, the extent to which the fusion protein bears the radiolabel is assessed in the test sample and compared with the extent to which the fusion protein bears the radiolabel in the control sample. If the detectable label is a first member of a binding pair (e.g. biotin), the second member of the pair (a binding partner) is added to the samples in order to detect the extent to which the fusion protein is bound by the reference D-peptide. This can be done directly or indirectly (e.g., by adding a molecule, such as an antibody or other moiety which binds the second member of the binding pair). Less of the label will be present on the fusion protein (N-helix coiled-coil cavity) if the candidate drug has inhibited (totally or partially) binding of the D-peptide to the semi-pocket. If binding occurs to a lesser extent in the test sample (in the presence of the candidate drug) than in the control sample (in the absence of the candidate drug), then the candidate drug is a drug that binds the N-helix coiled-coil cavity of RSV F1.

The soluble model described herein, or a variant thereof, e.g., in the D-enantiomer, is useful to identify molecules or compounds which are members of a library or collection and bind the N-helix coiled-coil of F1. For example, a library or collection of molecules or compounds, such as a phage display library, can be screened with the soluble model in the D-enantiomer to identify members that bind the semi-pocket. The mirror image of the soluble model, or a variant thereof, is used as the target molecule. As used herein, the terms “D-enantiomer of a polypeptide” and “D-peptide” refer to the exact mirror image of the molecule in the natural handedness. Thus, for amino acid residues that contain a second chiral center, such as Ile and Thr, the exact mirror image of the naturally-occurring amino acid residue is used to create the D version of the polypeptide. Also as used herein, the terms “D-amino acids” and “L-amino acids” are both meant to include the non-chiral amino acid glycine. The D version of the soluble model can be immobilized to a solid surface, such as by addition of one member of a binding pair (e.g., biotin) to it and addition of the other member of the pair (e.g., streptavidin) to the solid surface. Binding of the two members results in immobilization of the D soluble model on the solid surface, such as for phage panning. A linker which is an enzyme recognition site (e.g., an amino acid linker such as Gly-Lys-Gly, in which an L-lysine residue is used) can be placed between the D soluble model sequence and the binding pair member (between the biotin and D soluble model) to provide an enzyme recognition site (here, a trypsin recognition site), so that bound phage can be eluted by a trypsin digestion, rather than by non-specific elution, such as acid addition. The phage display library can be a library of L-amino acid peptides of any appropriate length fused to an appropriate phage gene. In one embodiment, it is a phage display library of L-amino acid peptides fused to the gIII gene of M13 phage. The peptides, in one embodiment, comprise 10 randomly encoded amino acid residues flanked by either a cysteine or a serine on both sides. Typically, several rounds of panning are carried out. D-soluble model-specific binding phage are identified. Phage that bind only the F1 region of the D-soluble model can be identified by post-panning assessment, such as by screening against wells that lack the antigen and then further testing against a panel of molecules. The mirror-image phage display method described herein has demonstrated the value of the soluble model and its D-enantiomers in identifying inhibitors of RSV entry that bind the F1 pocket.

The D-version of the soluble model can be used in a similar manner with other biologically encoded libraries, to discover other pocket-binding molecules that are not subject to enzymatic degradation by natural enzymes. For example, other phage-display libraries can be used to identify new D-peptide inhibitors (e.g., with a different number of residues between the flanking Cys residues and/or with randomly encoded amino acid residues outside the regions flanked by cysteine residues and/or with more than two cysteine residues). Strategies for encoding peptide libraries without phage (e.g., in which the encoding mRNA is attached to the peptide) can be used to identify D-peptide inhibitors. RNA or DNA libraries can be used (e.g., with SELEX methods) to identify L-ribose- or L-deoxyribose-based RNA or DNA aptamers, respectively, that bind to the hydrophobic pocket and are not substrates for natural nucleases (see e.g., Williams et al., PNAS, 74:11285 (1997)).

Although the versions of the soluble model of natural L-handedness can also be used in a similar manner with biologically encoded libraries, the most likely applications will be with other, non-biologically encoded libraries. For example, chemical combinatorial libraries on beads (of the one-bead, one-compound variety) can be screened with labeled soluble model (e.g., radioactive or with a chromophore) to identify beads containing molecules that bind to the soluble model. As another example, beads to which the soluble model had been previously attached can be incubated with a mixture of potential pocket-binding molecules (e.g., a mixture of chemicals, or a natural product extract). The soluble model (bound to the beads) can then be separated from the mixture, washed, and then subjected to conditions (e.g., organic solvent, low pH, high temperature) that elute molecules bound to the soluble model on the beads. The eluted molecules (i.e., potential semi-pocket-binding molecules) could be identified by analytical chemistry methods (e.g., HPLC, mass spectrometry).

Drugs identified by the methods described above are then further tested for their ability to inhibit (totally or partially) HRSV F1 function (membrane fusion) and, thus entry into cells, using further in vitro assays known to those of skill in the art, and/or in vivo assays in appropriate animal models or in humans.

One embodiment of the present invention is a method of identifying a drug that binds the N-helix coiled-coil of HRSV F1, particularly the N-helix coiled-coil pocket. The method comprises combining a candidate drug to be assessed for its ability to bind the N-helix coiled-coil pocket of HRSV F1 and peptide which comprises a soluble, trimeric coiled-coil and a sufficient portion of the N-peptide of HRSV F1 to include the HRSV F1 semi-pocket, under conditions appropriate for presentation of the HRSV F1 semi-pocket for binding by a molecule or compound (e.g., a drug) and determining whether the candidate drug binds the HRSV F1 semi-pocket. If binding of the candidate drug with the HRSV F1 semi-pocket occurs, the candidate drug is a drug which binds the N-helix coiled-coil pocket of HRSV F1. Optionally, binding of the candidate drug can be assessed in the assay as described above, except that a peptide that binds the N-helix coiled-coil semi-pocket (a peptide previously identified as one which binds the pocket) is combined with the candidate drug and the peptide. In this competitive assay, binding of the candidate drug to the N-helix coiled-coil semi-pocket is assessed in the presence of a known binding moiety (a molecule or compound which binds the semi-pocket). If binding of the candidate drug occurs in the presence of the known binding moiety, the candidate drug is a drug which binds the N-helix coiled-coil semi-pocket with sufficient affinity to successfully compete with the known binding moiety. The fusion protein used in this embodiment comprises a soluble, trimeric version of a coiled-coil, such as a soluble, trimeric version of the coiled-coil region of a protein (e.g., a non-RSV protein, such as that of GCN4 or GCN4-pIQI, although an RSV protein can be used) and a sufficient portion of the N-peptide of HRSV F1 to include the HRSV F1 semi-pocket. The fusion protein can comprise a soluble, trimeric version of the coiled-coil of any protein, provided that when it is in the fusion protein with the HRSV component, the HRSV semi-pocket is presented in such a manner that it is available for binding. It can be, for example, that of GCN4-pIQI, GCN4-pII, Moloney Murine Leukemia Virus (Mo-MLV) or the ABC heterotrimer. In one embodiment, the fusion protein is in the D-form. In another embodiment, the fusion protein is in the natural L-handedness.

In the competitive assay format, any peptide known to bind the N-helix coiled-coil cavity can be used as the known binding moiety. Also, any non-peptide pocket-binding molecule can be used in the competitive assay format. The competitive assay can be performed in solution, on a bead, or on a solid surface.

In one embodiment, the candidate drug is detectably labeled and binding of the candidate drug to the HRSV F1 N-helix coiled-coil is determined by detecting the presence of the detectable label on the HRSV F1 N-helix coiled-coil (as a result of binding of the labeled candidate drug to the N-helix coiled-coil). Detection of the label on the helix coiled-coil pocket of the soluble model is indicative of binding of the candidate drug to the N-helix coiled-coil semi-pocket and demonstrates that the candidate drug is a drug which binds the N-helix coiled-coil semi-pocket. If the labeled candidate drug is detected on the fusion protein, the candidate drug is a drug which binds the N-helix coiled-coil semi-pocket.

In another embodiment of the method of identifying a drug that binds the N-helix coiled-coil pocket of the HRSV F1, a soluble model that presents the semi-pocket in such a manner that it is available for binding by a drug is combined with a candidate drug and whether binding of the candidate drug with the N-helix coiled-coil of the soluble model occurs is determined. If binding occurs, the candidate drug is a drug which binds the pocket. Here, too, a competitive assay format can be used. The components of the competition assay can be labeled, with any of a variety of detectable labels, including fluorophore/quencher combinations. The candidate drug can be labeled, as described above, with any of a variety of detectable labels. The components of the soluble model (fusion protein) used in this embodiment and the competing moiety which is used in a competitive assay format can also be as described above.

The present invention also relates to a method of producing a drug that binds the N-helix coiled-coil pocket of HRSV F1. In one embodiment, the method is carried out as follows: A soluble model that presents the N-helix coiled-coil pocket of HRSV F1 or a fusion protein which comprises a soluble, trimeric coiled-coil (e.g., of a protein, such as a non-RSV protein, such as GCN4-pIQI, GCN4-pII, Mo-MLV, ABC heterotrimer or an RSV protein) is combined with a candidate drug to be assessed for its ability to bind the N-helix coiled-coil pocket of HRSV F1 and inhibit entry into cells, under conditions appropriate for presentation of the HRSV F1 semi-pocket for binding by a drug. Whether the candidate drug binds the HRSV F1 semi-pocket is determined, wherein if binding of the candidate drug to the N-helix coiled-coil semi-pocket of HRSV F1 occurs, the candidate drug is a drug which binds the N-helix coiled-coil cavity of HRSV F1. In this embodiment, the fusion protein comprises a soluble, trimeric coiled-coil (e.g., of a protein such as a non-RSV protein, such as a soluble, trimeric coiled coil of GCN4, GCN4-pIQI, GCN4-pII, Mo-MLV, ABC heterotrimer or an RSV protein) and a sufficient portion of the N-peptide of HRSV F1 to include the HRSV F1 coiled-coil semi-pocket portion. The soluble model, as described herein, can be used in this method; the D enantiomer of the soluble model can also be used (e.g., in mirror-image phage applications). The ability of the drug produced to inhibit HRSV entry into cells is assessed as described herein. It can be further assessed in an appropriate animal model or in humans.

The invention also relates to a method of producing a drug that binds the N-helix coiled-coil pocket of HRSV F1. The method comprises: producing or obtaining a soluble model of the N-helix coiled-coil semi-pocket of HRSV F1; combining a candidate drug (a molecule or compound) to be assessed for it ability to bind the N-helix coiled-coil semi-pocket of HRSV F1 and the soluble model of the N-helix coiled-coil pocket of HRSV F1 and determining whether the candidate drug binds the N-helix coiled-coil pocket of HRSV F1. If the candidate drug binds the N-helix coiled-coil pocket of HRSV F1, the candidate drug is a drug which binds the N-helix coiled-coil pocket of HRSV F1; as a result, a drug which binds the N-helix coiled-coil semi-pocket of HRSV F1 is produced. Alternatively, a drug that binds the N-helix coiled-coil semi-pocket of HRSV F1 and inhibits entry of HRSV into cells can be produced by a method comprising: producing or obtaining a soluble model of the N-helix coiled-coil semi-pocket of HRSV F1, as described herein; combining the soluble model and a candidate drug to be assessed for its ability to bind the N-helix coiled-coil semi-pocket of HRSV F1; determining whether the candidate drug binds the N-helix coiled-coil pocket of the soluble model (fusion protein), wherein if binding occurs, the candidate drug is a drug which binds the N-helix coiled-coil of HRSV F1; and assessing the ability of the drug which binds the N-helix coiled-coil to inhibit HRSV entry into cells, wherein if the drug inhibits HRSV entry into cells, it is a drug which binds the N-helix coiled-coil semi-pocket of HRSV F1 and inhibits HRSV entry into cells. Its ability to inhibit HRSV entry into cells can be assessed in vitro or in vivo (e.g. in an appropriate animal model or in humans). The soluble model can be a peptide which comprises a soluble, trimeric coiled-coil, such as that of a protein (e.g., GCN4-pIQI) and a sufficient portion of the N-peptide of HRSV F1 to include the HRSV F1 semi-pocket.

A drug or molecule which binds or fits into a cavity or semi-pocket on the surface of N57, can be used alone or in combination with other drugs (as part of a drug cocktail) to prevent or reduce HRSV infection of humans. A drug designed or formed by a method described herein is also the subject of this invention. Such drugs can be obtained, for example, by linking two or more pocket-binding molecules (drugs) via an appropriate linker (e.g., a linker of amino aicd residues or other chemical moieties) to increase the effectiveness of inhibition. The pocket-binding molecules that are linked can be the same or different.

Also the subject of this invention is a method of treating an individual infected with HRSV or at risk of being infected with HRSV, in order to reduce the extent of infection or to prevent infection. In the method, a drug which fits into, lines or binds a cavity or semi-pocket on N57 is administered to the individual, alone or in combination with other drugs.

A further subject of this invention is an immunogen based on a molecule with coordinates as described herein which is used to produce antibodies (human, humanized) that bind the N57 cavity or pocket and, thus, prevent N57/C45 interaction and inhibit F1 activity. For example, the N-peptide core or soluble model described herein can be used, in known methods, to produce polyclonal or monoclonal antibodies, which can be administered to an individual. Alternatively, an individual (e.g., a human infected with RSV or at risk or being infected) can be immunized with the N-peptide core or the soluble model. The individual will, as a result, produce antibodies which will bind the N57 pocket or cavity and prevent or reduce F1 activity. Thus, this invention also relates to a vaccine to reduce or prevent F1 function (and, as a result, HRSV infection). Compounds and molecules (drugs) identified as described herein inhibit (partially or totally) entry of HRSV into cells, and thus are useful therapeutically in uninfected individuals (humans) and infected individuals (e.g., to prevent or reduce infection in an uninfected individual, to reduce or prevent further infection in an infected individual) and as research reagents both to study the mechanism of F1-induced membrane fusion and to assess the rate of viral clearance by an individual and as reagents to discover or develop other compounds and molecules (drugs) that inhibit entry of HRSV into cells.

In particular, the present invention relates to an isolated antibody (monoclonal antibody, such as a human or a humanized monoclonal antibody) that binds to a peptide, wherein the peptide comprises a soluble, trimeric form of a coiled-coil and a sufficient portion of the N-peptide region of HRSV F1 to comprise the amino acid residues that form part or all of the N-helix coiled-coil of HRSV F1 and the antibody binds to the sufficient portion of the peptide. In one embodiment, the peptide to which the isolated antibody binds comprises a sufficient portion of the N-peptide region of HRSV F1 to comprise the amino acid residues that form the semi-pocket of the N-helix coiled-coil of HRSV F1. In a particular embodiment, the isolated antibody is a neutralizing antibody.

The present invention also relates to a method of producing antibodies that bind to the N-helix coiled-coil region of HRSV F1. In the method, a peptide which presents the N-helix coiled-coil region of HRSV F1 as a properly folded trimer is introduced into an individual, whereby the individual produces antibodies that a bind the N-helix coiled-coil region of HRSV F1 and antibodies that bind the N-helix coiled-coil region of HRSV F1 are removed from the individual. The antibodies can be removed from the individual by obtaining blood from the individual and separating the antibodies from the blood, thereby producing antibodies that bind to the N-helix coiled-coil region of HRSV F1.

The present invention also relates to a method of obtaining human monoclonal antibodies that bind the N-helix coiled-coil region of HRSV F1, comprising obtaining a sample of blood, B-cells, spleen or bone marrow from an individual who has produced antibodies to HRSV; producing clones of cells that produce monoclonal antibodies from the sample; contacting the clones or tissue culture supernatants of the clones with a peptide which presents the N-helix coiled-coil region of HRSV F1 as a properly folded trimer, under conditions whereby antibodies that bind the N-helix coiled-coil region of HRSV F1 bind the peptide, whereby antibodies produced by the clones that bind the N-helix coiled-coil region of HRSV F1 bind the peptide, thereby obtaining human monoclonal antibodies that bind the N-helix coiled-coil region of HRSV F1. In one embodiment, the individual is HRSV infected or immunized against HRSV. The method can further comprise separating antibodies from the peptides to which they are bound. In addition, in the method, the peptide can comprise a soluble trimeric form of a coiled-coil and a sufficient portion of the N-peptide region of RSV F1 to comprise the amino acid residues that form the semi-pocket of the N-helix coiled-coil of RSV F1.

Also encompassed by the present invention is a method of providing protection against HRSV infection, comprising administering to an individual in need of protection against HRSV infection neutralizing antibodies described herein.

The present invention also relates to a pharmaceutical composition comprising isolated neutralizing antibodies described herein in a pharmaceutically acceptable carrier.

The drugs can be administered by a variety of route(s), such as orally, nasally, intraperitoneally, intramuscularly, vaginally or rectally. In each embodiment, the drug is provided in an appropriate carrier or pharmaceutical composition. For example, a semi-pocket-binding drug can be administered in an appropriate buffer, saline, water, gel, foam, cream or other appropriate carrier. A pharmaceutical composition comprising the drug and, generally, an appropriate carrier and optional components, such as stabilizers, absorption or uptake enhancers, flavorings and/or emulsifying agents, can be formulated and administered in therapeutically effective dose(s) to an individual (uninfected or infected with HRSV). In one embodiment, drugs which bind the N-helix coiled-coil of F1 are administered (or applied) as microbicidal agents and interfere with viral entry into cells. For example, a drug or drugs which bind(s) the HRSV cavity can be included in a composition which is applied to or contacted with a mucosal surface, such as the vaginal, rectal or oral mucosa. The composition comprises, in addition to the drug, a carrier or base (e.g., a cream, foam, gel, other substance sufficiently viscous to retain the drug, water, buffer) appropriate for application to a mucosal surface. The drug can be applied to a mucosal surface, such as by application of a foam, gel, cream, water or other carrier containing the drug. Alternatively, it can be applied by means of a vaginal or rectal suppository which is a carrier or base which contains the drug or drugs and is made of a material which releases or delivers the drug (e.g., by degradation, dissolution, other means of release) under the conditions of use (e.g., vaginal or rectal temperature, pH, moisture conditions). Such compositions can also be administered orally (e.g., swallowed in capsule, pill, liquid or other form) and pass into an individual's blood stream. In all embodiments, controlled or time release (gradual release, release at a particular time after administration or insertion) of the drug can be effected by, for example, incorporating the drug into a composition which releases the drug gradually or after a defined period of time. Alternatively, the drug can be incorporated into a composition which releases the drug immediately or soon after its administration or application (e.g., into the vagina, mouth or rectum). Combined release (e.g., release of some of the drug immediately or soon after insertion, and over time or at a particular time after insertion) can also be effective (e.g., by producing a composition which is comprised of two or more materials: one from which release or delivery occurs immediately or soon after insertion and/or one from which release or delivery is gradual and/or one from which release occurs after a specified period). For example, a drug or drugs which bind the HRSV semi-pocket can be incorporated into a sustained release composition such as that taught in U.S. Pat. No. 4,707,362. Release of the drug(s) can occur immediately, gradually or at a specified time, as described above. As a result, they make contact with and bind HRSV and reduce or prevent viral entry into cells.

As described above, Applicants have provided the identity of amino acid residues which form the semi-pocket into which amino acid residues of the F1 C-peptides fit. Thus, they have defined target amino acid residues which can be mutated or modified, individually or jointly, to further assess the structural basis for interaction between the two peptides, identify amino acid residues essential for the two to fit together and design or identify molecules or compounds which inhibit/prevent the two helices from fitting together and, thus, inhibit or prevent F1 membrane—fusion activity.

Like other enveloped viruses, HRSV has a glycoprotein-containing envelope that permits the virus to bind to the target cell and achieve cell entry. HRSV carries three surface glycoproteins: F, G, and SH (Collins et al., Respiratory Syncytial Virus. In Fields Virology Third edit. (Fields, B. N., Knipe, D. M. & Howley, P. M., eds.), pp. 1313-1351. Lippincott—Raven Publishers, Philadelphia 1996). The F protein mediates the fusion of the viral membrane with the cellular membrane. The G protein has been shown to be physically associated with F (Arumugham et al., Arch Virol, 105(1-2): 65-79 (1989)), and mediates the attachment of virus to the target cell. The SH protein has been found dispensable for virus growth. Of these three proteins, the F protein is the most attractive therapeutic target, as it is highly conserved, essential for viral entry, and is the major virus neutralization antigen.

The F protein is synthesized as a precursor, F0 (67 kDa), which is then proteolytically cleaved to two subunits F1(47 kDa) and F2 (21 kDa) that are disulfide-linked. The membrane-anchored subunit F1 has the fusion peptide at the very N-terminus and the transmembrane segment proximal to the C-terminus. The fusion peptide portion is very hydrophobic and is supposed to insert into the target membrane during the fusion process. Two heptad repeat regions characterized by coiled coil-forming sequences have been identified in F1 subunit by computational approaches (Chambers et al., J Gen Virol, (71):3075-3080 (1990); Buckland et al., J Gen Virol 73(Pt 7): 1703-7 (1992); Singh et al., J. Mol. Biol. 290:1031-1041 (1999)). One heptad repeat region is adjacent to the fusion peptide and the other is next to the transmembrane segment. These two regions are separated by ˜270 residues. Such arrangement of the structural elements is highly conserved for the Paramyxoviridae family F proteins (Lamb & Kolakofsky, Paramyxoviridae: The Viruses and Their Replication. In Fields Virology Third edit. (Fields, B. N., Knipe, D. M. & Howley, P. M., eds.), pp. 1177-1204. Lippincott—Raven Publishers, Philadelphia (1996)).

Heptad repeat regions have been characterized by structural studies in membrane-fusion proteins of a variety of different viruses including hemagglutinin HA of influenza virus (Wilson et al., Nature 289: 366-373 (1981); Bullough et al., Nature 371: 37-43 (1994)), gp41 of human and simian immunodeficiency virus (HIV and SIV) (Chan et al., Cell 89: 263-273 (1997); Weissenhorn et al., Nature 387: 426-430 (1997)), TM of Moloney murine leukemia virus (MoMLV) (Fass et al., Nature Struct. Biol. 3: 465-469 (1996)), GP2 of Ebola virus (Malashkevich et al., Proc Natl Acad Sci U S A 96(6): 2662-7 (1999); Weissenhorn et al., Proc. Natl. Acad. Sci. USA 95: 6032-6036 (1998)), gp21 of human T cell leukemia virus type-1 (HTLV-1) (Kobe et al., Proc Natl Acad Sci U S A 96(8): 4319-24 (1999)), and F1 of simian parainfluenza virus 5 (SV5) (Baker et al., Mol Cell 3(3): 309-19 (1999)). These heptad-repeat sequences all form trimeric hairpin-like structures, with the C-terminal residues in the outer layer fold back towards the N-terminal end of the inner coiled coil. A consequence of hairpin formation is that the fusion peptide is brought close to the transmembrane segment. Given that the fusion peptide inserts into the cellular membrane and the transmembrane segment is anchored in the viral membrane, the likely role of the hairpin structure is to facilitate the apposition of the viral and cellular membranes, the fusion-peptide and transmembrane regions. Hence, the trimer-of-hairpins structure presumably corresponds to the fusion-active state.

Extensive studies have suggested that viral fusion proteins undergo a conformational change to become fusogenic when exposed to the appropriate triggering signals (Hernandez et al., Annu Rev Cell Dev Biol 12: 627-61 (1996)). Upon low pH triggering, influenza HA uses a “spring-loaded mechanism” to switch from the kinetically trapped metastable native conformation to the more stable fusion-active conformation (Wilson et al., Nature 289: 366-373 (1981); Carr & Kim, Cell 73: 823-832 (1993); Bullough et al., Nature 371: 37-43 (1994). For HIV envelope protein gp120/gp41, upon receptor binding, the protein appears to transit through at least three different conformations: the native form, prehairpin intermediate, and the fusogenic hairpin form (Chan & Kim, Cell 93: 681-684 (1998)).

In the prehairpin intermediate conformation, the inner coiled-coil regions are supposed to be transiently exposed. It therefore provides an opportunity to use exogeous inhibitors to target the inner coiled coil and block the hairpin formation. In the case of HIV studies, the C-terminal heptad-repeat region of gp41, referred to as C peptide, can effectively inhibit HIV infection (Kilby et al., Nature Med. 4: 1302-1307 (1998); Chan et al., Proc. Natl. Acad. Sci. USA 95: 15613-15617 (1998)). It has been proposed that C peptides function in a dominant-negative manner by binding to the transiently exposed inner coiled coil in the pre-hairpin intermediate and consequently blocking the formation of the fusion-active hairpin structures (Chan & Kim, Cell 93: 681-684 (1998)). Recently small D-peptides as well as chemical compounds that specifically target the inner coiled coil and inhibit viral fusion have been identified by library approaches (Eckert et al., Cell 99(1): 103-15 (1999); Ferrer et al., Nat Struct Biol 6(10): 953-60 (1999)). This may lead to a new class of drugs that can block viral entry.

Similar approaches would be effective in identifying inhibitors of HRSV infection. The presence of the two heptad-repeat regions in the HRSV F1 proteins indicates that the F1 proteins adopt a trimer-of-hairpins structure in their fusion-active conformations. The core structure of another Paramyxoviridae family F protein, the SV5 F protein, has been shown to be a trimer of hairpins with remarkable similarity to that of HIV gp41 (Baker et al., Mol Cell 3(3): 309-19 (1999)). Since the HRSV and SV5 F protein share similar structural arrangement, it is likely that they adopt a similar fold. Pre-hairpin intermediate conformation seems to exist for both the HRSV and SV5 F1 protein, as peptides corresponding to their C-terminal heptad-repeat regions have been shown to inhibit viral infection (Joshi et al., Virology 248(1): 20-34 (1998); Lambert et al., Proc Natl Acad Sci U S A 93(5): 2186-91 (1996)). By analogy to HIV C peptides, these peptides likely act in a dominant-negative manner to prevent the formation of final fusion-active structures and thereby inhibit viral entry.

A high-resolution structure of the HRSV F1 core would contribute to the understanding of the fusion mechanism of the HRSV F protein and benefit the development of potent inhibitors of viral entry. Here the characterization of the two heptad repeat regions of HRSV F1 protein is reported. Two peptides, N57 and C45, which correspond to the two heptad repeats of HRSV F1 protein, are found to form a stable trimer of heterodimers. N57/C45 complex was crystallized and its X-ray structure was determined at 2.3 Å resolution. The structure confirms that the complex indeed forms a trimer of hairpins. A close look of the N57/C45 interface indicates the presence of a long groove on the surface of the central coiled coil which serves as a good drug target.

The present invention is illustrated by the following examples, which are not intended to be limiting in any way.

### EXAMPLES

The materials and methods described below were used in the examples that follow.

Materials and Methods

Gene Construction and Purification of recRSV-1. Using optimal codons for E. coli expression (Makrides, S. C. (1996) Microbiol. Rev. 60, 512-538), a synthetic gene sequence denoted recRSV-1 was constructed that encodes recRSV-1 (residues 153-209 and 476-524 of HRSV (strain RSS-2; Swiss-Prot accession #P 1209) connected by a glycine-rich linker). (See FIG. 1.) A Factor Xa cleavage site was incorporated upstream of the HRSV coding sequence. The constructed gene was inserted into the BamHI-HindIII restriction site of the hexahistidine expression vector pQE9 (Qiagen, Valencia, Calif.). The resulting plasmids, denoted as pRSV-1, were transformed into E.coli XL1-Blue competent cells for protein expression. Cells were grown in Luria-Bertani medium to an optical density of 0.6 at 600 nm. Protein expression was then induced with 1 mM isopropyl-β-D-thiogalactopyranoside (IPTG) and cells were harvested after 3 hours.

Cells were lysed in 6M GuHCl and the lysate was clarified by centrifugation at 15,000 rpm for 30′. The recombinant protein was purified by nickel-nitrilotriacetic acid (Ni-NTA) metal-affinity chromatography, followed by reverse-phase HPLC (Waters, Milford, Mass.) using a Vydac C18 preparative column (Vydac, Hesperia, Calif.) with a water/acetonitrile gradient of 0.1%/min in the presence of 0.1% trifluoro acetic acid (TFA). The mass of the purified protein was verified by mass spectrometry on a Voyager Elite MALDI-TOF mass spectrometer (PerSeptive Biosystems, Framingham, Mass.) and lyophilized. The protein was resuspended in ultra-pure water and dialyzed against Factor Xa cleavage buffer (50 mM Tris-HCl, pH 8.0, 100 mM NaCl, 2 mM CaCl₂). To remove the His tag, Factor Xa was added at a 1:500 weight:weight ratio of protease to tagged protein and the reaction incubated for 2 days at room temperature. The cleavage mixture was then purified by reverse-phase HPLC on a Vydac C18 preparative column. Peak fractions containing recRSV-1 were verified by mass spectrometry and lyophilized.

Proteolysis.

All proteolysis reactions were performed with 1 mg/ml protein and 0.1 mg/ml protease in phosphate-buffered saline (PBS) buffer, pH 7.4, at room temperature and were quenched with 2 mM phenylmethylsulfonyl fluoride (final concentration). Proteolysis samples were analyzed by reverse phase HPLC connected to an LCQ electrospray mass spectrometer (Finnigan MAT, San Jose, Calif.). Fragments were assigned by matching observed masses with a list of possible fragment masses predicted by a computer program (E. Wolf & P.S.K. http://www.wi.mit.edu/kim/computing.html). All assigned fragments were within 1 dalton of their predicted values.

Circular Dichroism (CD) Spectroscopy.

CD spectra were measured at protein concentration 10 μM in PBS buffer using an AVIV 62 DS spectrometer (Aviv Associates, Lakewood, N.J.), as described (Fass, D. & Kim, P. S., Curr. Biol., 5:1377-1383 (1995).

Sedimentation Equilibrium Analysis.

Sedimentation equilibrium analysis was performed on a Beckman XL-A analytical ultracentrifuge at 20° C. at rotor speeds of 15,000 rpm and 20,000 rpm. Three protein samples at a different concentration of 10 μM, 50 μM, and 100 μM were dialyzed against PBS buffer overnight. Data analysis was performed as described (Laue, T. M. et al. (1992) in: Analytical Ultracentrifugation in Biochemistry and Polymer Science (Harding, S. E. et al. eds.), pp. 90-125. Royal Society of Chemistry, Cambridge).

Purification, Crystallization and Structure Determination of the HRSV-N57/C45 Trimer.

The HRSV-N57 and HRSV-C45 peptides were generated by trypsin digestion of recRSV-1 protein and purified to homogeneity by reverse-phase HPLC on a Vydac C8 preparative column. The purified HRSV peptides were lyophilized and dissolved in water and 10 mM Tris-HCl (pH 8.5), respectively. Equimolar amounts of HRSV-N57 and HRSV-C45 were mixed and the complex separated from the free peptide and aggregated species by gel filtration on a Sephacryl S-100 HR column (Pharmacia, Uppsala, Sweden) in buffer (10 mM Tris-HCl, pH 8.5, 50 mM NaCl). The purified HRSV-N57/C45 complex was concentrated to 10 mg/ml.

Crystals were obtained using the hanging drop method by equilibrating 2_{—}1 drops (protein solution mixed 1:1 with reservoir solution) against a reservoir solution containing 24-26% polyethylene glycol 4000, 200 mM Tris-HCl, pH 8.5, and 300 mM Li2SO4. The crystals belong to the space group P1 (a=67.9 Å, b=71.5 Å, c=76.5 Å, (α=81.3°, β=73.8°, γ=60.7°). There are four trimers in one asymmetric unit and the solvent content is ˜46%. For data collection, crystals were directly mounted in nylon loops (Hampton Research, Inc.) from the drop and flash frozen in liquid nitrogen. Diffraction data were collected at 100 K at the Howard Hughes Medical Institute Beamline X4A at Brookhaven National Laboratory using a Quantum-4 CCD detector. Diffraction intensities were integrated using the DENZO and SCALEPACK software (Otwinowski, Z. (1993). Oscillation data reduction program. In Data Collection and Processing (Sawer, L., Isaacs, N. & Bailey, S., ed.), pp. 55-62, SERC, Daresbury Laboratory, Warrington, England), and reduced to structure factors with the program TRUNCATE from the CCP4 program suite (CCP4, Acta. Crystallogr. D 50: 760-763 (1994)). The structure of the N57/C45 complex was solved by molecular replacement using the program AMoRe (Navaza, J., Acta. Crystallogr. D50: 760-763 (1994)). A polyserine model derived from the SV5 N1/C1 trimer (Baker et al., Mol. Cell. 3(3): 309-19(1999)) was used as a search model. Initially only three solutions were found. After applying solvent flattening, histogram matching and non-crystallographic averaging with the program DM (Kowtan, K., Newsletter on Protein Crystallography, 31: 34-38 (1994)), the electronic density map was interpretable in most regions. Three HRSV-N57/C45 trimer molecules were built into the density using the program O (Jones et al., (1991) Acta. Crystallogr., D47: 110-119). After a few cycles of refinement, the R_{free} value remained high and the existence of unassigned density patches was suggestive of a fourth molecule. A molecular replacement search with a partially refined HRSV-N57/C45 trimer molecule readily produced the fourth trimer in the unit cell. Crystallographic refinement of the structure (Table) was done with the CNS programs (Brunger, A. T. (1996) (Yale University Press, New Haven)) (reflections beyond 2.3 Å were not used due to poor completeness). Noncrystallographic symmetry restraints were applied during the first few cycles of refinement, but were then removed and not used in the final refinement. The quality of coordinates was examined by PROCHECK (Laskowski, R. A., et al., J. Appl. Crystallogr., 26:283-291). No residues were in disallowed regions, and 94.5% were in the most favored regions of the Ramachandran plot.

Results

HRSV-N57 and HRSV-C45 Peptides form a Stable Trimeric α-helical Complex

LeamCoil-VMF, a program for identifying coiled-coil-like regions in viral fusion proteins, predicts such regions in the HRSV F1 protein (Singh, M. et al. (1999) J. Mol. Biol. 290, 1031-1041). The first region (residues 156-201) is near the putative fusion peptide and the second region (residues 488-516) is adjacent to the transmembrane segment (FIG. 1).

To study the regions predicted by LeamCoil-VMF a recombinant protein, recRSV-1, corresponding to residues 153-209 and residues 476-524, of HRSV F protein connected by a glycine-rich linker was constructed (see Methods) (FIG. 1). CD spectroscopy indicates that recRSV-1 is highly helical (˜70% helix content) and extremely thermostable (the midpoint of thermal denaturation [Tₘ]˜90° C. in the presence of 4 M GuHCl, pH 7.4); recRSV-1 forms a trimer in PBS buffer as judged by sedimentation equilibrium analysis.

Proteolysis of the recRSV-b 1 protein reveals a protease-resistant core containing the entire HR-N and HR-C regions predicted by LearnCoil-VMF. Digestion of recRSV-1 by trypsin generates two major fragments readily separated by reverse-phase HPLC. Mass spectrometry analysis unambiguously assigns these as a N-terminal fragment corresponding to residues 153-209, denoted HRSV-N57, and a C-terminal fragment corresponding to residues 476-520, denoted HRSV-C45 (FIG. 1).

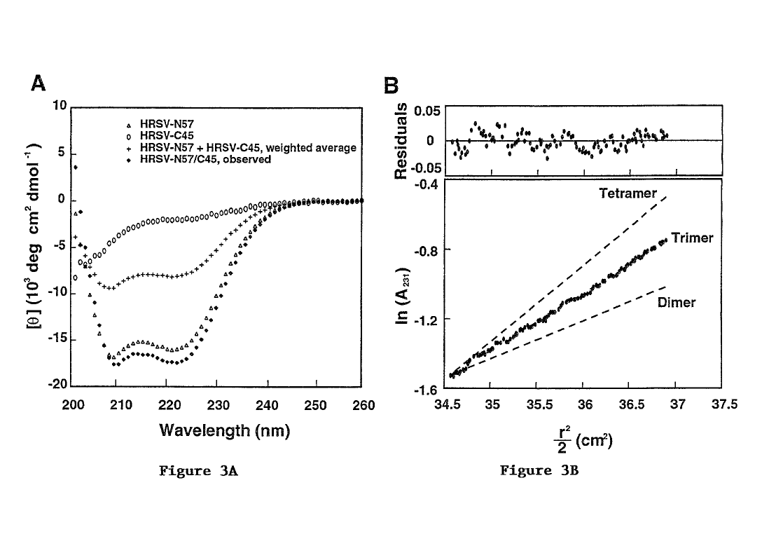

The isolated HRSV-N57 peptide folds into an α-helical structure in PBS (>40% helix content) as determined by CD, but does not form a monodispersed species as judged by sedimentation equilibrium analysis. The apparent Tₘ of HRSV-N57 varies (40-50° C. at 10 μM in PBS), presumably due to aggregation. In contrast, the isolated HRSV-C45 peptide is unfolded and the CD signal does not show a thermal unfolding transition. The CD spectrum of an equimolar mixture of the HRSV-N57 and HRSV-C45 peptides (FIG. 3A) shows a substantially higher α-helical signal than the weighted average of the spectra of the individual peptides, indicating a major conformational change upon association of the two peptides. The HRSV-N57/C45 complex has an apparent Tₘ of 88° C. at 10 μM in PBS. Sedimentation equilibrium centrifugation clearly indicates that in HRSV-N57/C45 complex the peptides are present in 3:3 ratio (FIG. 3B). These results are in agreement with those of Lambert and colleagues (Lawless-Delmedico, M. K. et al. (2000) Biochemistry 39, 11684-11695).

HRSV-N57/C45 can be further trimmed by proteinase K to generate smaller fragments denoted HRSV-N51 (residues 157-207) and HRSV-C39 (residues 482-520) (FIG. 1). Proteinase K is not sequence specific and therefore its proteolytic fragments are expected to better represent the boundaries of a well-folded domain. The HRSV-N51/C39 complex has an apparent Tₘ of 88° C. at 10 μM in PBS, the same as that of HRSV-N57/C45. Thus, the extra-terminal residues in HRSV-N57 and HRSV-C45 do not seem to contribute to the stability of the core complex.

Crystal Structure of HRSV-N57/C45

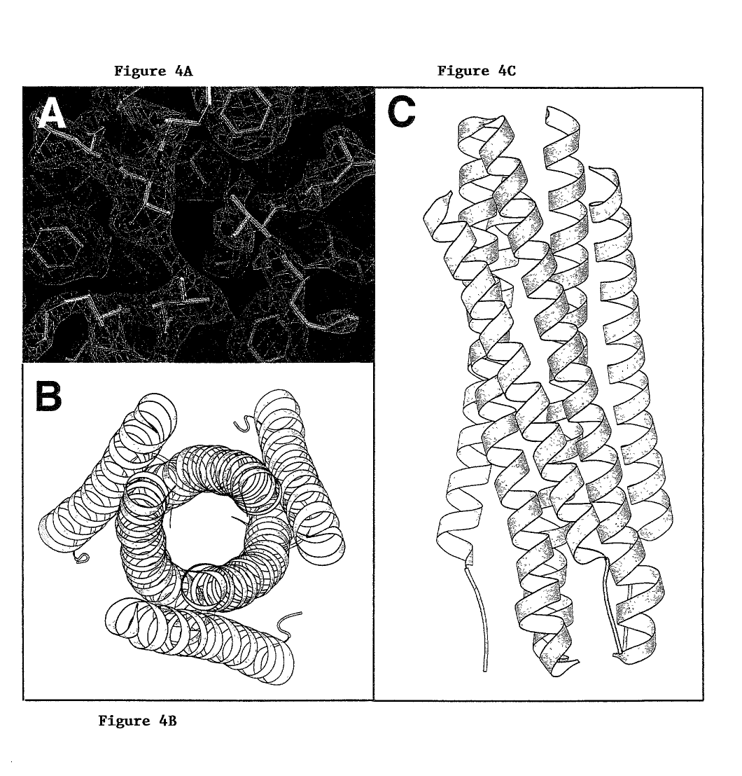

Crystals of HRSV-N57/C45 contain four trimers of HRSV-N57/C45 heterodimers per unit cell, giving rise to 12 independently refined heterodimers. Both the final 2Fₒ-F_{c} map and simulated annealing omit map are readily interpretable (FIG. 4A). However, some terminal residues, including 153-159, 208-209, 476-479, and 517-520, are disordered to a different degree among different chains. Although it is possible that this variation is due to the differences in local lattice contact environments, these terminal residues are more likely to be intrinsically flexible, especially in solution, since they are removed in the proteinase K-digested HRSV-N57/C45.

We focused our structural analysis on the well-defined regions, including residues 160-207 in HRSV-N57 and residues 480-516 in HRSV-C45. For these regions, the structural differences between the 12 copies of the HRSV-N57/C45 heterodimer are small (e.g., the average root-mean-square difference [RMSD] in C_{α} positions between any pair of heterodimers is 0.34 Å).

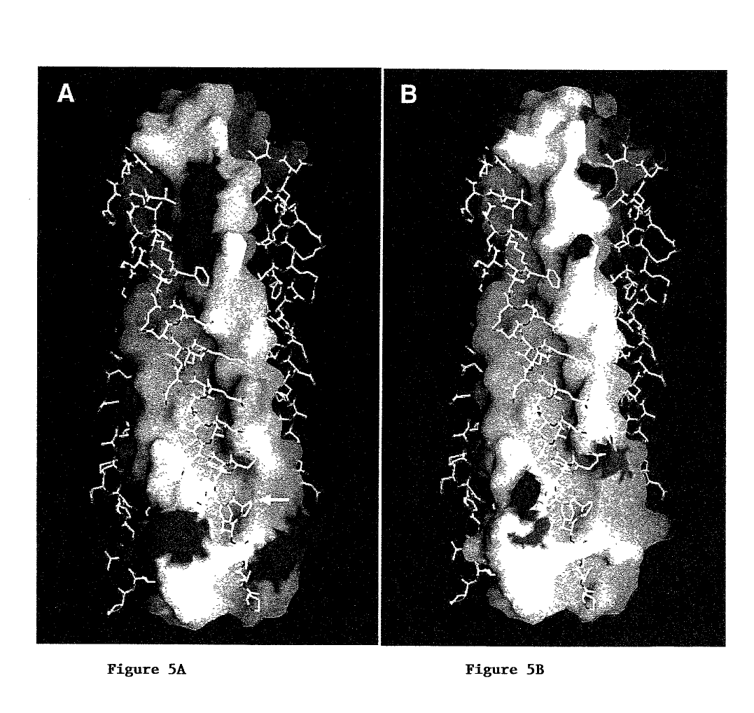

HRSV-N57/C45 forms a rod-shaped, six-helix bundle of approximately 68 Å in length and 27 Å in diameter (FIGS. 4B,C). The HRSV-N57 peptides form a three-stranded coiled coil. Three HRSV-C45 peptides pack in an antiparallel manner against long, hydrophobic grooves formed on the surface of the HRSV-N57 core. This packing arrangement would put the fusion peptide segment, located immediately before HRSV-N57, and transmembrane segment, located directly after HRSV-C45, in close proximity. The interface between the HRSV-N57 core and HRSV-C45 Approximately 2900 Å² of solvent-accessible surface area are buried at the interface between the HRSV-N57 and each HRSV-C45 peptide. Twenty amino acids from each HRSV-C45 peptide and 26 amino acids from two adjacent HRSV-N57 peptides contribute to the interface interaction. These residues are highly conserved among different HRSV strains, including 20 HRSV isolates and 7 bovine respiratory syncytial virus (BRSV) isolates (analysis based on sequences available in GenBank and Swiss-Prot as of September 2000) (FIG. 5A). Most of the amino-acid differences between strains are limited to conservative mutations.

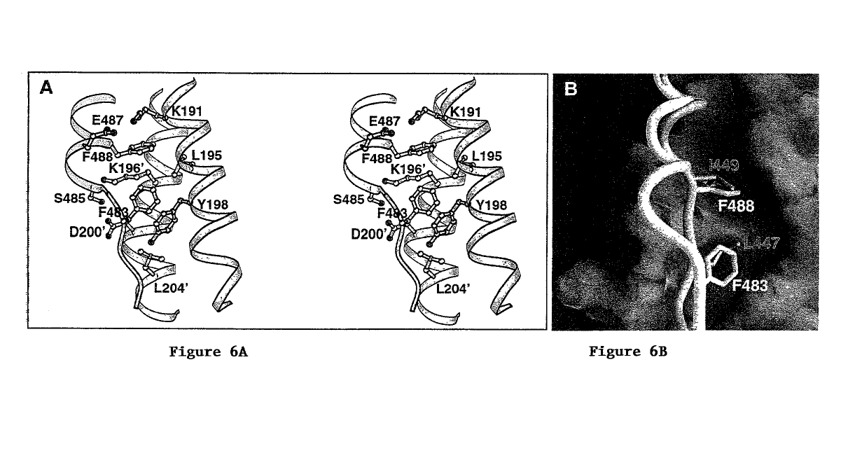

Various features of the deep groove on the surface of the HRSV-N57 trimer (FIG. 5B) suggest that this groove is a potentially attractive drug target. The carboxy-terminal end of the groove features a hydrophobic cavity. Within the cavity region, the majority of contacts are between six cavity-lining residues from the HRSV-N57 peptides (K191, L195, and Y198 from one chain, and K196, D200, and L204 from another) and two aromatic residues, F483 and F488, from the HRSV-C45 peptide (FIGS. 6A,B). A patch in the middle of the hydrophobic groove (FIG. 5B) is enriched with polar residues. Quite a few hydrogen bonds are observed at the corresponding HRSV-N57/C45 interface. For example, S509 Oγ of the HRSV-C45 peptide forms an elaborate hydrogen-bond network with T174 Oγ1, A170 O, and N175 Nδ2 from the HRSV-N57 helices. The abundance of hydrogen-bond donor/acceptors in the groove is likely to greatly enhance binding specificity due to the requirement of chemical complementarity. Thus, this cavity and groove are potentially good binding sites for small molecule inhibitors (Eckert, D. M. et al. (1999) Cell 99, 103-115; Chan D. C. et al. (1998) Proc. Natl. Acad. Sci. USA 95, 15613-15617).

Comparison with SV5 F1 Trimer Core and Other Viral Fusion Proteins

Among paramyxovirus fusion proteins, only the structure of the SV5 F1 trimer core has been determined (Baker, K. A. (1999) Mol. Cell 3, 09-319). In general, the F1 core from HRSV and SV5 adopt a similar fod, despite a low sequence identity between the two proteins (18.4% for the entire protein sequence and 19.1% for the heptad-repeat regions). These structures can be superimposed with an RMSD of 2.1 Å between all C_{α} atoms. The central N-peptide cores superimpose closely, with an RMSD of 0.65 Å, whereas the C-peptides are more divergent, with an RMSD of 2.9 Å.

A stutter or increase in spacing pattern (3-4-4-4-3) for heptad repeats, instead of the regular 3-4-3-4-3, has been observed in the SV5 F1 trimer core (Baker, K. A. (1999) Mol. Cell 3, 309-319). A similar stutter is observed within the last two heptad repeats in the HRSV N57 coiled-coil core. This supports the prediction by Baker et al. that the 3-4-4-4-3 pattern will be conserved among paramyxovirus F proteins since it can maintain hydrophobic residues within the coiled-coil (Baker, K. A. (1999) Mol. Cell 3, 09-319). However, since the stutter in HRSV N57 occurs at the end of the peptide helix, structural irregularities resulting from helix end-effects cannot be ruled out.

It has been suggested that the C-peptides of HRSV and other paramyxoviruses will have a similar main chain conformation as observed in the SV5 crystal structure (Baker, K. A. (1999) Mol. Cell 3, 309-319). However, results described herein show that the HRSV C-peptide has one additional helical turn not observed in the SV5 C-peptide. As a consequence, the position of the C-peptide residues that pack into the hydrophobic cavity (FIG. 6B) is shifted in HRSV compared with SV5, and such a packing arrangement is not readily predicted based on primary sequence alignment of the two proteins.

Discussion

The structure of HRSV N57/C45 adds to the repertoire of viral fusion proteins that have been shown to form a trimer-of-hairpins motif. The remarkable similarity between the HRSV F1 and HIV-1 gp41 core structures, as well as similar C-peptide inhibition phenomena, suggest a conserved mechanism of fusion. Similar to HIV-1 gp41, HRSV F1 likely undergoes a series of conformational changes to become fusion active. The distinct conformational states proposed for HIV-1 gp41 (Chan, D. C. & Kim, P. S. (1998) Cell 93, 681-684), including the native state, the prehairpin intermediate, and fusion-active state, may also apply to HRSV F1. Electron microscopy studies of the full-length F protein reveal two distinct conformations, one cone-shaped and the other lollipop-shaped, which may represent the native and fusion-active states, respectively (Calder, L. J. et al. (2000) Virology 271, 122-131). The HRSV-N57/C45 structure presented here likely corresponds to the fusion-active hairpin conformation as the hairpin formation is structurally coupled to the apposition of viral and cellular membranes.

The existence of the prehairpin intermediate conformation for the HRSV F protein is strongly supported by the observation that peptides corresponding to the HRSV HR-C regions can efficiently inhibit viral fusion. Presumably these HR-C peptides function by binding to the exposed HR-N regions, thereby blocking the conformational transition to the fusion-active form. The exceptional stability of HRSV-N57/C45 may explain the potency of exogenous C-peptide inhibitors.

If small, oral, bioavailable molecules that disrupt hairpin formation are identified, they may be effective drugs against viral infection. In the case of HIV-1, a strategy to block hairpin formation has been developed to find D-peptide inhibitors of viral entry that bind to a hydrophobic pocket on the surface of the central coiled coil consisting of HIV-1 gp41 N-peptides (Eckert, D. M. et al. (1999) Cell 99, 103-115). Described herein is identification of a well-defined groove on the surface of the central coiled coil of the HRSV N57/C45 complex structure. A strategy similar to the one used with HIV-1 may be applicable to HRSV.

Finally, the HRSV-N57/C45 structure may provide a new direction in vaccine development against HRSV infection. Antibodies from vaccine trials using purified, native, full-length F protein have very low neutralizing activities compared with those generated by live HRSV (Collins, P. L., et al. (1996) in Fields Virology, Third ed. (Fields, B. N., et al., eds.) pp.1313-1351. Lippincott—Raven Publishers, Philadelphia). One suggested strategy for eliciting neutralizing HIV-1 antibodies is to target transient intermediates or fusion-competent conformations (Eckert, D. M. et al. (1999) Cell 99, 103-115; LaCasse, R. A. et al. (1999) Science 283, 357-362) terminal coiled coil observed in the current structure might be formed in a prehairpin intermediate analogous to that found in HIV-1, and thus may be a viable target for anti-fusion antibodies. A neutralizing antibody against HRSV has been mapped to the N-terminal coiled-coil region (Langedijk, H. P. et al. (1998) Arch. Virol. 143, 313-320) and its ability to target a prehairpin intermediate of HRSV can be assessed using known methods.

TABLE
X-ray data collection and refinement statistics

| Data collection | |
|---|---|
| Resolution range, Å | 20.0-2.20 |
| Observed reflections | 119,905 |
| Unique reflections | 56,973 |
| Completeness, % | 91.2 |
| | (64.7)^{a} |
| R_{merge}^{b} | 0.041 |
| | (0.269)^{a} |
| Refinement | |
| Resolution range, Å | 10.0-2.30 |
| Protein nonhydrogen atoms | 8073 |
| Water molecules | 888 |
| R_{cryst}^{c} | 0.233 |
| R_{free}^{c} | 0.286 |
| RMSD from ideal geometry | |
| Bond lengths, Å | 0.008 |
| Bond angles, ° | 1.2 |
| Average B-factor, Å² | 45.4 |

^{a}Values in parentheses correspond to highest resolution shell 2.28 to 2.20 Å.
^{b}R_{merge} = ΣΣⱼ|Iⱼ (hkl) − <I(hkl)>|/ΣΣⱼ|<I(hkl)>|, where Iⱼ is the intensity measurement for reflection hkl and <I> is the mean intensity over j reflections.
^{c}R_{cryst} (R_{free}) = Σ||F_{obs}(hkl)| − |F_{calc}(hkl)||/Σ|F_{obs} (hkl)|, where F_{obs} and F_{calc} are observed and calculated structure factors, respectively. No σ-cutoff was applied. 10% of the reflections were excluded from refinement and used to calculate R_{free}.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of identifying a drug that inhibits the RSV membrane fusion machinery by inhibiting interactions between the N57 peptide trimer and the C45 peptide trimer of RSV F1, comprising:
(a) combining RSV F1 N57 peptide trimer, RSV F1 C45 peptide trimer and a drug to be assessed for its ability to inhibit interaction between the two trimers, to produce a combination;
(b) maintaining the combination under conditions appropriate for interactions to occur between N57 peptide trimers and C45 peptide trimers; and
(c) assessing whether interactions occurred between N57 peptide trimers and C45 peptide trimers, wherein if interactions between the N57 peptide trimer and the C45 peptide trimer did not occur in the presence of the drug or occurred to a lesser extent in the presence of the drug than in its absence, the drug is a drug that inhibits the RSV membrane fusion machinery.

2. The method of claim 1 wherein RSV is HRSV and in step (c) the interaction assessed is packing of amino acid residues or peptides of the C45 peptide trimer into a highly conserved semi-pocket on the N57 peptide trimer.

3. The method of claim 2 wherein the interaction assessed is packing of amino acid residues or peptides of C45 into a hydrophobic groove formed by two N57 peptide chains which form the semipocket, wherein the semipocket is lined by K191, L195 and Y198 from a first N57 chain and K196, D200 and L204 from a second N57 chain.

4. A method of producing a drug which inhibits interaction of two components of the core of fusion-active HRSV envelope F1, wherein the two components are referred to as N57 peptide trimer and C45 peptide trimer, respectively, comprising identifying a compound or designing a compound which fits into a hydrophobic groove formed by two N57 peptide chains which form the semipocket, wherein the semipocket is lined by K191, L195 and Y198 from a first N57 chain and K196, D200 and L204 from a second N57 chain.

5. The method of claim 4 wherein N57 peptide trimer and C45 peptide trimer are recombinantly produced.

6. A method of producing a drug which inhibits interaction of N57 peptide trimer with C45 peptide trimer, wherein N57 peptide trimer and C45 peptide trimer comprise the core of fusion-active HRSV envelope F1, comprising identifying a compound or designing a compound which fits into a hydrophobic groove formed by two N57 peptide chains which form a semipocket, wherein the semipocket is lined by K191, L195 and Y198 from a first N57 chain and K196, D200 and L204 from a second N57 chain.

7. A compound which inhibits interaction of N57 peptide trimer of the α-helical domain of HRSV F1 which is the core or fusion active F1 with C45 peptide trimer of the α-helical domain.

8. The compound of claim 8 wherein the compound fits into a hydrophobic groove formed by two N57 peptide chains which form the semipocket, wherein the semipocket is lined by K191, L195 and Y198 from a first N57 chain and K196, D200 and L204 from a second N57 chain.

9. A peptide which comprises a soluble, trimeric form of a coiled coil and a sufficient portion of the N-peptide region of HRSV F1 to comprise the amino acid residues which form a semi-pocket of the N-helix coiled coil of HRSV F1.

10. The peptide of claim 9 wherein the peptide is selected from the group consisting of an L peptide and a D-peptide.

11. A method of identifying a drug that interferes with the formation of a complex between C45 peptide and N57 peptide, comprising:
(a) combining a candidate drug to be assessed for its ability to interfere with formation of a complex between C45 peptide and N57 peptide, under conditions appropriate for formation of a complex between C45 peptide and N57 peptide, thereby forming a test sample; and
(b) determining whether formation of a complex between C45 peptide and N57 peptide is inhibited, wherein if formation of the complex is inhibited, the candidate drug is a drug that interferes with formation of the complex whereby a drug that interferes with formation of the complex is identified.

12. The method of claim 11 wherein a control sample is formed by combining C45 peptide and N57 peptide, under the same conditions as the conditions under which the test sample is formed in (a); formation of a complex between C45 peptide and N57 peptide is determined and the extent to which the complex is formed in the test sample is compared with the extent to which the complex is formed in the control sample, wherein if the complex is formed to a lesser extent in the test sample than in the control sample, the candidate drug is a drug that interferes with formation of the complex, whereby a drug that interferes with formation of the complex is identified.

13. A method of eliciting an immune response in an individual, comprising introducing into the individual a peptide comprising a trimeric form of a coiled-coil region of a protein and a sufficient portion of the N-peptide region of RSV F1 to comprise the amino acid residues which form part or all of the N-helix coiled-coil of RSV F1 and the peptide is present in a pharmaceutically acceptable carrier.

14. The method of claim 13 wherein the peptide is introduced into the individual by a route of administration selected from the group consisting of: intramuscularly, intraperitoneally, orally, nasally and transdermally.

15. A method of identifying a compound or molecule which binds the N-helix coiled-coil semi-pocket of HRSV F1 envelope protein, wherein the compound or molecule to be assessed is referred to as a candidate inhibitor, comprising:
(a) combining a D-peptide which binds the N-helix coiled-coil cavity, a fusion protein which is a soluble model which presents the N-helix coiled-coil cavity and a candidate inhibitor, under conditions appropriate for binding of the D-peptide to the N-helix coiled-coil cavity, thereby producing a test sample;
(b) determining the extent to which binding occurs of the D-peptide to the N-helix coiled-coil semi-pocket in the test sample; and
(c) comparing the extent of binding determined in the N-helix coiled-coil semi-pocket in a control sample, wherein the control sample is the same as the test sample except that the control sample does not include the candidate inhibitor and is maintained under the same conditions appropriate for binding of the D-peptide to the N-helix coiled-coil semi-pocket as is the test sample, wherein if the extent of binding in the test sample is less than the extent of binding in the control sample, the candidate inhibitor is a compound or molecule which binds the N-helix coiled-coil semi-pocket of RSV F1 envelope protein.

16. A method of eliciting an immune response in an individual, comprising introducing into the individual a fusion protein comprising a soluble, trimeric form of a coiled-coil and a sufficient portion of the N-peptide region of HRSV F1, to comprise the amino acid residues which form the pocket of the N-helix coiled-coil of HRSV F1, wherein the fusion protein is present in a pharmaceutically acceptable carrier.

17. A method of identifying a drug that binds the N-helix coiled-coil semi-pocket of HRSV F1 comprising:
(a) combining: (1) a candidate drug to be assessed for its ability to bind the N-helix coiled-coil semi-pocket of HRSV F1 and; (2) a fusion protein which comprises a trimeric version of the coiled-coil region of a protein and a sufficient portion of the N-peptide of HRSV F1 to include the HRSV F1 semi-pocket, under conditions appropriate for presentation of the HRSV F1 semi-pocket for binding by a drug; and
(b) determining whether the candidate drug binds the HRSV F1 semi-pocket, wherein if binding occurs, the candidate drug is a drug which binds the N-helix coiled-coil semi-pocket of HRSV F1.

18. The method of claim 17 wherein in (a), a peptide which binds the N-helix coiled-coil semi-pocket of HRSV F1 is combined with the candidate drug and the fusion protein and in (b), whether the candidate drug binds the HRSV F1 semi-pocket is determined in the presence of the peptide which binds the N-helix coiled-coil semi-pocket of RSV F1.

19. A method of identifying a drug that binds the N-helix coiled-coil semi-pocket of HRSV F1 comprising:
(a) combining: (1) a soluble model that presents the N-helix coiled-coil semi-pocket of HRSV F1 in such a manner that it is available for binding by a drug and (2) a candidate drug, which is to be assessed for its ability to bind the N-helix coiled-coil semi-pocket; and
(b) determining whether the candidate drug binds the N-helix coiled coil semi-pocket of the soluble model, wherein if binding occurs, the candidate drug is a drug which binds the N-helix coiled-coil semi-pocket of HRSV F1.

20. A method of producing a drug that binds the N-helix coiled-coil semi-pocket of HRSV F1 and inhibits HRSV entry into cells, comprising:
(a) combining (1) a candidate drug to be assessed for its ability to bind the N-helix coiled-coil semi-pocket of HRSV F1 and inhibit HRSV entry into cells and (2) a fusion protein which comprises a trimeric version of the coiled-coil region of a protein and a sufficient portion of the N-peptide of HRSV F1 to include the HRSV F1 semi-pocket, under conditions appropriate for presentation of the HRSV F1 semi-pocket for binding by a drug;
(b) determining whether the candidate drug binds the HRSV F1 semi-pocket, wherein if binding of the candidate drug to the N-helix coiled-coil semi-pocket of HRSV F1 occurs, the candidate drug is a drug which binds the N-helix coiled-coil semi-pocket of HRSV F1, whereby a drug which binds the N-helix coiled-coil semi-pocket of HRSV F1 is produced; and
(c) assessing the ability of the drug produced in (b) to inhibit HRSV entry into cells, wherein if the drug inhibits HRSV entry into cells, it is a drug which binds the N-helix coiled-coil semi-pocket of HRSV F1 and inhibits HRSV entry into cells.

21. A method of producing a soluble model of the N-helix coiled-coil semi-pocket of HRSV F1, comprising producing a fusion protein comprising: (a) a soluble, trimeric form of a coiled-coil and (b) a sufficient portion of the N-peptide region of HRSV F1 to comprise the amino acid residues which form the pocket of the N-helix coiled-coil of HRSV F1.

22. A method of producing a drug that binds the N-helix coiled-coil semi-pocket of HRSV F1 comprising:
(a) producing or obtaining a soluble model of the N-helix coiled-coil semi-pocket of HRSV F1;
(b) combining: (1) a candidate drug to be assessed for its ability to bind the N-helix coiled-coil semi-pocket of HRSV F1 and (2) the soluble model of the N-helix coiled-coil semi-pocket of HRSV F1; and
(c) determining whether the candidate drug binds the N-helix coiled-coil semi-pocket of HRSV F1, wherein if the candidate drug binds the N-helix coiled-coil semi-pocket of HRSV F1, the candidate drug is a drug which binds the N-helix coiled-coil semi-pocket of HRSV F1, whereby a drug which binds the N-helix coiled-coil semi-pocket of HRSV F1 is produced.

23. The method of claim 22 wherein the soluble model is a fusion protein which comprises a trimeric version of the coiled-coil region of a protein and a sufficient portion of the N-peptide of HRSV F1 to include the HRSV F1 semi-pocket.

24. A method of producing a drug that binds the N-helix coiled-coil semi-pocket of HRSV F1 and inhibits its entry into cells, comprising:
(a) producing or obtaining a soluble model of the N-helix coiled-coil semi-pocket of HRSV F1;
(b) combining: (1) a candidate drug to be assessed for its ability to bind the N-helix coiled-coil semi-pocket of HRSV F1 and (2) the soluble model of the N-helix coiled-coil semi-pocket of HRSV F1;
(c) determining whether the candidate drug binds the N-helix coiled-coil semi-pocket of HRSV F1, wherein if the candidate drug binds the N-helix coiled-coil semi-pocket of HIV gp41, the candidate drug is a drug which binds the N-helix coiled-coil semi-pocket of HRSV F1, whereby a drug which binds the N-helix coiled-coil semi-pocket of HRSV F1 is produced and;
(d) assessing the ability of the drug produced in (c) to inhibit HRSV entry into cells, wherein if the drug inhibits HRSV entry into cells, it is a drug which binds the N-helix coiled-coil semi-pocket of HRSV F1 and inhibits HRSV entry into cells.

25. The method of claim 24 wherein the soluble model is a fusion protein which comprises a trimeric version of the coiled-coil region of a protein and a sufficient portion of the N-peptide of HRSV F1 to include the HRSV F1 semi-pocket

26. An isolated antibody that binds to a peptide, wherein the peptide comprises a soluble, trimeric form of a coiled-coil and a sufficient portion of the N-peptide region of HRSV F1 to comprise the amino acid residues that form part or all of the N-helix coiled-coil of HRSV F1 and the antibody binds to the sufficient portion of the peptide.

27. The isolated antibody of claim 26 wherein the peptide comprises a sufficient portion of the N-peptide region of HRSV F1 to comprise the amino acid residues that form the semi-pocket of the N-helix coiled-coil of HRSV F1.

28. The isolated antibody of claim 27 which is a monoclonal antibody.

29. The isolated antibody of claim 28 which is a human monoclonal antibody.

30. The isolated antibody of claim 26, wherein the antibody is a neutralizing antibody.

31. The isolated antibody of claim 30, wherein the peptide comprises a sufficient portion of the N-peptide region of HRSV F1 to comprise the amino acid residues that form the semi-pocket of the N-helix coiled-coil of HRSV F1.

32. The isolated antibody of claim 31 which is a monoclonal antibody.

33. The isolated antibody of claim 32 which is a human monoclonal antibody.

34. The isolated antibody of claim 32, wherein the monoclonal antibody is humanized.

35. An isolated antibody that binds the coiled-coil region of HRSV F1 as presented by a peptide which presents the N-helix coiled-coil region of HRSV F1 as a properly folded trimer.

36. The isolated antibody of claim 35, wherein the antibody is a neutralizing antibody.

37. The isolated antibody of claim 36, wherein the peptide comprises a sufficient portion.
